(19) United States
(12) Patent Application Publication
(10) Pub. No.: US 2004/0180409 A1
(43) Pub. Date: Sep. 16, 2004

(54) TLT-1, a novel platelet-associated receptor and uses therefor

(76) Inventors: Daniel McVicar, Charles Town, WV (US); A. Valance Washington, Fair Field, PA (US); Laura Quigley, Frederick, MD (US)

Correspondence Address: Peter F. Corless, EDWARDS & ANGELL, LLP, P.O. Box 55874, Boston, MA, 02205, US

(21) Appl. No.: 10/802,441

(22) Filed: Mar. 16, 2004

Related U.S. Application Data
(60) Provisional application No. 60/455,370, filed on Mar. 16, 2003.

(51) Int. Cl. C07K 14/705; C07H 21/04

(52) U.S. Cl. 435/69.1; 530/350; 435/320.1; 435/325; 536/23.5

(57) ABSTRACT

The invention provides isolated nucleic acids molecules, designated TLT-1 nucleic acid molecules, which encode novel inhibitory receptors expressed on platelets. The invention also provides antisense nucleic acid molecules, recombinant expression vectors containing TLT-1 nucleic acid molecules, host cells into which the expression vectors have been introduced, and nonhuman transgenic animals in which a TLT-1 gene has been introduced or disrupted. The invention still further provides isolated TLT-1 proteins, fusion proteins, antigenic peptides and anti-TLT-1 antibodies. Diagnostic methods utilizing compositions of the invention are also provided.

### RELATED APPLICATIONS

This application claims the benefit of U.S. Provisional Application Serial No. 60/455,370, filed Mar. 16, 2003, the entire contents of which are incorporated herein by this reference.

### BACKGROUND OF THE INVENTION

The triggering receptors expressed on myeloid cells (TREMs) are an emerging family of activating receptors expressed on various cells of the myeloid lineage (Bouchon, A. et al. (2000) J. Immunol. 164:4991-4995; Bouchon, A. et al. (2001) J. Exp. Med. 194:1111-1122; Daws, M. R. et al. (2001) Eur. J. Immunol. 31:783-791; Chung, D. H. et al. (2002) Eur. J. Immunol. 32:59-66). The TREMs represent a loose cluster (150 kb) on mouse chromosome 17, and the cluster's genomic organization is highly conserved on human chromosome 6 (FIG. 5). Although the family members possess only 30% amino acid identity, each member consists of a leader sequence, single V-set Ig domain, short cytoplasmic tail, and transmembrane domain containing a positively charged residue, suggesting interaction with a signaling polypeptide (Daws, M. R. et al. (2001) Eur. J. Immunol. 31:783-791; Chung, D. H. et al. (2002) Eur. J. Immunol. 32:59-66). Biochemical analysis has demonstrated that of the four TREM sequences described to date, TREMs 1, 2, and 3 associate with the activating signaling chain DAP 12, and TREM 4 is predicted to as well (Bouchon, A. et al. (2000) J. Immunol. 164:4991-4995; Bouchon, A. et al. (2001) J. Exp. Med. 194:1111-1122; Daws, M. R. et al. (2001) Eur. J. Immunol. 31:783-791; Chung, D. H. et al. (2002) Eur. J. Immunol. 32:59-66; Bouchon, A. et al. (2001) Nature 410:1103-1107). Recently, Bouchon et al. uncovered the importance of this family in the regulation of multiple facets of the immune response (Bouchon, A. et al. (2000) J. Immunol. 164:4991-4995; Bouchon, A. et al. (2001) J. Exp. Med. 194:1111-1122; Bouchon, A. et al. (2001) Nature 410:1103-1107). These studies defined TREM 1 as an important mediator of septic shock (Bouchon, A. et al. (2000) J. Immunol. 164:4991-4995; Bouchon, A. et al. (2001) Nature 410:1103-1107; Nathan, C and Ding, A. (2001) Nat. Med. 7:530-532; Cohen, J. (2001) Lancet 358:776-778) and TREM 2 as playing a unique role in dendritic cell maturation and, therefore, T-cell priming (Bouchon, A. et al. (2001) J. Exp. Med. 194:1111-1122; Bachmann, M. F. (2002) Trends Immunol. 23:10). Taken together, these data demonstrate the intriguing potential for receptors of the TREM family to be key regulators of both the innate and adaptive immune response. Despite the recent advances in TREM immunobiology, TREM ligands and mode of regulation remain ill-defined, and there exists a need in the art for agents and methods that can regulate the TREMs.

Platelets, also referred to as “blood platelets” or “peripheral blood platelets”, are small cells that lack a nucleus but have a highly organized cytoskeleton, unique cell-surface receptors, and specialized secretory granules. Human blood contains nearly a trillion platelets, which respond to blood vessel injury by changing shape, secreting granule contents, and aggregation (Italiano, J. E., Jr. et al. (1999) J. Cell Biol. 147:1299-1312). These responses cause blood clotting to aid repair of injury and stop bleeding, but can also cause unwanted clots that lead to tissue ischemia and/or infarction, including stroke and heart attack.

Platelets are produced through the terminal differentiation of megakaryocytes. Each mature megakaryocyte produces and releases hundreds of platelets into circulation (Kaufinan et al. (1965) Blood 26:720-728; Harker and Finch (1969) J. Clin. Invest. 48:963-974; and Trowbridge et al. (1984) Clin. Phys. Physiol. Meas. 5:145-156). Megakaryocytes, which make up about <0.1 % of all cells in the bone marrow (Italiano et al. (1999) supra), are polyploid cells whose size and DNA content correlate directly with the circulating platelet mass (Ebbe and Stohlman (1965) Blood 26:20-34). Mature megakarypcytes assemble a unique set of organelles, including alpha granules, dense bodies, and an extensive system of internal membranes (Shivdasani, R. A. (2001) Stem Cells 19:397-407). Differentiated megakarycytes extrude long cytoplasmic processes (“proplatelets”) that serve as the immediate precursors of circulating platelets (Choi, E. S. et al. (1995) Blood 85:402-413; Cramer, E. M. et al. (1997) Blood 89:2336-2346; Norol, F. et al. (1998) Blood 91:830-843). Megakaryocyte and platelet differentiation is controlled by a number of transcription factors, including GATA-1, FOG-1, and NF-E2 (Shivdasani (2001) supra), as well as factors such as thrombopoietin.

Given the importance of platelets in blood clotting and wound healing, as well as their involvement in many disorders such as stroke and heart disease, there exists a need in the art for agents and methods that can modulate platelet production and/or function.

### SUMMARY OF THE INVENTION

The present invention is based, at least in part, on the discovery of a novel inhibitory receptor within the TREM locus, referred to herein as TLT-1 (TREM-like transcript-1) nucleic acid and protein molecules. The TLT-1 nucleic acid and protein molecules of the present invention are useful as modulating agents in regulating a variety of cellular processes, e.g., blood clotting and immune response. Accordingly, in one aspect, this invention provides isolated nucleic acid molecules encoding TLT-1 proteins or biologically active portions thereof, as well as nucleic acid fragments suitable as primers or hybridization probes for the detection of TLT-1-encoding nucleic acids.

In one embodiment, a TLT-1 nucleic acid molecule of the invention is at least 50%, 55%, 60%, 65%, 70%, 75%, 80%, 81%, 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to the nucleotide sequence (e.g., to the entire length of the nucleotide sequence) shown in SEQ ID NO:1, 3, 4, 6, 18, 20, 21, 23, 24, or 26, or a complement thereof.

In a preferred embodiment, the isolated nucleic acid molecule includes the nucleotide sequence shown in SEQ ID NO:1, 3, 4, 6, 18, 20, 21, 23, 24, or 26, or a complement thereof. In another embodiment, the nucleic acid molecule includes SEQ ID NO:3 and nucleotides 1-21 of SEQ ID NO: 1. In another embodiment, the nucleic acid molecule includes SEQ ID NO:3 and nucleotides 988-1220 of SEQ ID NO: 1. In yet another embodiment, the nucleic acid molecule includes SEQ ID NO:6 and nucleotides 934-936 of SEQ ID NO:4. In another embodiment, the nucleic acid molecule includes SEQ ID NO:20 and nucleotides 1-27 of SEQ ID NO:18. In another embodiment, the nucleic acid molecule includes SEQ ID NO:20 and nucleotides 325-422 of SEQ ID NO:18. In another embodiment, the nucleic acid molecule includes SEQ ID NO:23 and nucleotides 1-21 of SEQ ID NO:21. In another embodiment, the nucleic acid molecule includes SEQ ID NO:23 and nucleotides 973-1205 of SEQ ID NO:21. In another embodiment, the nucleic acid molecule includes SEQ ID NO:26 and nucleotides 1-21 of SEQ ID NO:24. In another embodiment, the nucleic acid molecule includes SEQ ID NO:26 and nucleotides 619-907 of SEQ ID NO:24. In another preferred embodiment, the nucleic acid molecule consists of the nucleotide sequence shown in SEQ ID NO:1, 3, 4, 6, 18, 20, 21, 23, 24, or 26.

In another embodiment, a TLT-1 nucleic acid molecule includes a nucleotide sequence encoding a protein having an amino acid sequence sufficiently identical to the amino acid sequence of SEQ ID NO:2, 5, 19, 22, or 25. In a preferred embodiment, a TLT-1 nucleic acid molecule includes a nucleotide sequence encoding a protein having an amino acid sequence at least 50%, 55%, 60%, 65%, 70%, 75%, 80%, 81%, 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to the entire length of the amino acid sequence of SEQ ID NO:2, 5, 19, 22, or 25.

In another preferred embodiment, an isolated nucleic acid molecule encodes the amino acid sequence of mouse or human TLT-1. In yet another preferred embodiment, the nucleic acid molecule includes a nucleotide sequence encoding a protein having the amino acid sequence of SEQ ID NO:2, 5, 19, 22, or 25. In yet another preferred embodiment, the nucleic acid molecule is at least 10, 20, 30, 40, 50, 100, 150, 200, 250, 300, 333, 336, 350, 400, 450, 487, 500, 519, 550, 600, 650, 700, 750, 800, 850, 900, 950, 1000, 1050, 1100, 1150, 1200, or more nucleotides in length. In a further preferred embodiment, the nucleic acid molecule is at least 10, 20, 30, 40, 50, 100, 150, 200, 250, 300, 333, 336, 350, 400, 450, 487, 500, 519, 550, 600, 650, 700, 750, 800, 850, 900, 950, 1000, 1050, 1100, 1150, 1200, or more nucleotides in length and encodes a protein having a TLT-1 activity (as described herein).

Another embodiment of the invention features nucleic acid molecules, preferably TLT-1 nucleic acid molecules, which specifically detect TLT-1 nucleic acid molecules relative to nucleic acid molecules encoding non-TLT-1 proteins. For example, in one embodiment, such a nucleic acid molecule is at least 10, 20, 30, 40, 50, 100, 150, 200, 250, 300, 333, 336, 350, 400, 450, 487, 500, 519, 550, 600, 650, 700, 750, 800, 850, 900, 950, 1000, 1050, 1100, 1150, 1200, 1210 or more nucleotides in length and hybridizes under stringent conditions to a nucleic acid molecule comprising the nucleotide sequence shown in SEQ ID NO:1, 3, 4, 6, 18, 20, 21, 23, 24, or 26, or a complement thereof.

In preferred embodiments, the nucleic acid molecules are at least 15 (e.g., 15 contiguous) nucleotides in length and hybridize under stringent conditions to the nucleotide molecules set forth in SEQ ID NO:1, 3, 4, 6, 18, 20, 21, 23, 24, or 26, or a complement thereof.

In other preferred embodiments, the nucleic acid molecule encodes a naturally occurring allelic variant of a polypeptide comprising the amino acid sequence of SEQ ID NO:2, 5, 19, 22, or 25, wherein the nucleic acid molecule hybridizes to a complement of a nucleic acid molecule comprising SEQ ID NO:1, 3, 4, 6, 18, 20, 21, 23, 24, or 26, respectively, under stringent conditions.

Another embodiment of the invention provides an isolated nucleic acid molecule which is antisense to a TLT-1 nucleic acid molecule, e.g., the coding strand of a TLT-1 nucleic acid molecule.

Another aspect of the invention provides a vector comprising a TLT-1 nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a TLT-1 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant TLT-1 proteins and polypeptides. In one embodiment, an isolated TLT-1 protein includes at least one or more of the following domains: a transmembrane domain, a signal peptide domain, and IgV domain a cysteine residue, an extracellular domain, a cytoplasmic domain, a polyproline-rich region, an ITIM, a PEST domain, and an O-glycosylation site.

In a preferred embodiment, a TLT-1 protein includes at least one or more of the following domains: a transmembrane domain, a signal peptide domain, and IgV domain, a cysteine residue, an extracellular domain, a cytoplasmic domain, a polyproline-rich region, an ITIM, a PEST domain, and an O-glycosylation site, and has an amino acid sequence at least about 50%, 55%, 60%, 65%, 67%, 68%, 70%, 75%, 80%, 81%, 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to the amino acid sequence of SEQ ID NO:2, 5, 19, 22, or 25.

In another preferred embodiment, a TLT-1 protein includes at least one or more of the following domains: a transmembrane domain, a signal peptide domain, and IgV domain, a cysteine residue, an extracellular domain, a cytoplasmic domain, a polyproline-rich region, an ITIM, a PEST domain, and an O-glycosylation site, and has a TLT-1 activity (as described herein).

In yet another preferred embodiment, a TLT-1 protein includes at least one or more of the following domains: a transmembrane domain, a signal peptide domain, and lgV domain, a cysteine residue, an extracellular domain, a cytoplasmic domain, a polyproline-rich region, an ITIM, a PEST domain, and an O-glycosylation site, and is encoded by a nucleic acid molecule having a nucleotide sequence which hybridizes under stringent hybridization conditions to a complement of a nucleic acid molecule comprising the nucleotide sequence of SEQ ID NO:1, 3, 4, 6, 18, 20, 21, 23, 24, or 26.

In another embodiment, the invention features fragments of the protein having the amino acid sequence of SEQ ID NO:2, 5, 19, 22, or 25, wherein the fragment comprises at least 5, 6, 7, 8, 9, 10, 15, 20, 25, 30, 35, 40, 45, 50, 75, 100, 125, 150, 175, 200, 225, 250, 275, 300, 305, 310, 311, 312, 313, 314, 315, 320, 321 amino acids (e.g., contiguous amino acids) of the amino acid sequence of SEQ ID NO:2, 5, 19, 22, or 25. In another embodiment, a TLT-1 protein has the amino acid sequence of SEQ ID NO:2, 5, 19, 22, or 25.

In another embodiment, the invention features a TLT-1 protein which is encoded by a nucleic acid molecule consisting of a nucleotide sequence at least about 50%, 55%, 60%, 65%, 70%, 75%, 80%, 81% 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to a nucleotide sequence of SEQ ID NO:1, 3, 4, 6, 18, 20, 21, 23, 24, or 26, or a complement thereof. This invention further features a TLT-1 protein which is encoded by a nucleic acid molecule consisting of a nucleotide sequence which hybridizes under stringent hybridization conditions to a complement of a nucleic acid molecule comprising the nucleotide sequence of SEQ ID NO: 1, 3, 4, 6, 18, 20, 21, 23, 24, or 26, or a complement thereof.

The proteins of the present invention or portions thereof, e.g., biologically active portions thereof, can be operatively linked to a non-TLT-1 polypeptide (e.g., heterologous amino acid sequences) to form fusion proteins. The invention further features antibodies, such as monoclonal or polyclonal antibodies, that specifically bind proteins of the invention, preferably TLT-1 proteins. In addition, the TLT-1 proteins or biologically active portions thereof can be incorporated into pharmaceutical compositions, which optionally include pharmaceutically acceptable carriers.

In another aspect, the present invention provides a method for detecting the presence of a TLT-1 nucleic acid molecule, protein, or polypeptide in a biological sample by contacting the biological sample with an agent capable of detecting a TLT-1 nucleic acid molecule, protein, or polypeptide such that the presence of a TLT-1 nucleic acid molecule, protein or polypeptide is detected in the biological sample.

In another aspect, the present invention provides a method for detecting the presence of TLT-1 activity in a biological sample by contacting the biological sample with an agent capable of detecting an indicator of TLT-1 activity such that the presence of TLT-1 activity is detected in the biological sample.

In another aspect, the invention provides a method for modulating TLT-1 activity comprising contacting a cell capable of expressing TLT-1 with an agent that modulates TLT-1 activity such that TLT-1 activity in the cell is modulated. In one embodiment, the agent inhibits TLT-1 activity. In another embodiment, the agent stimulates TLT-1 activity. In one embodiment, the agent is an antibody that specifically binds to a TLT-1 protein. In another embodiment, the agent modulates expression of TLT-1 by modulating transcription of a TLT-1 gene or translation of a TLT-1 mRNA. In yet another embodiment, the agent is a nucleic acid molecule having a nucleotide sequence that is antisense to the coding strand of a TLT-1 mRNA or a TLT-1 gene.

In one embodiment, the methods of the present invention are used to treat a subject having a disorder characterized by aberrant or unwanted TLT-1 protein or nucleic acid expression or activity by administering an agent which is a TLT-1 modulator to the subject. In one embodiment, the TLT-1 modulator is a TLT-1 protein. In another embodiment the TLT-1 modulator is a TLT-1 nucleic acid molecule. In yet another embodiment, the TLT-1 modulator is a peptide, peptidomimetic, or other small molecule. In a preferred embodiment, the disorder characterized by aberrant or unwanted TLT-1 protein or nucleic acid expression is a platelet-associated disorder, e.g., a bleeding or clotting disorder.

The present invention also provides diagnostic assays for identifying the presence or absence of a genetic alteration characterized by at least one of (i) aberrant modification or mutation of a gene encoding a TLT-1 protein; (ii) mis-regulation of the gene; and (iii) aberrant post-translational modification of a TLT-1 protein, wherein a wild-type form of the gene encodes a protein with a TLT-1 activity.

In another aspect the invention provides methods for identifying a compound that binds to or modulates the activity of a TLT-1 protein, by providing an indicator composition comprising a TLT-1 protein having TLT-1 activity, contacting the indicator composition with a test compound, and determining the effect of the test compound on TLT-1 activity in the indicator composition to identify a compound that modulates the activity of a TLT-1 protein.

Other features and advantages of the invention will be apparent from the following detailed description and claims.

### BRIEF DESCRIPTION OF THE DRAWINGS

FIG. 1 depicts the nucleotide sequence of mouse TLT-1 (SEQ ID NO:1).

FIG. 2 depicts the amino acid sequence of mouse TLT-1 (SEQ ID NO:2).

FIG. 3 depicts the nucleotide sequence of human TLT-1 (SEQ ID NO:4).

FIG. 4 depicts the amino acid sequence of human TLT-1 (SEQ ID NO:5).

FIG. 5 depicts the genomic organization of TREM-like genes in the human and mouse. A schematic of the relationship between the human TREM cluster and the mouse TREM cluster is shown at the top. The exon structure of the TLT-1 gene is shown at the bottom. The asterisk denotes the premature stop in the smaller mRNA species caused by the alternative splice event detected by RT-PCR in RAW264.7 cells and dendritic cell cultures.

FIG. 6 depicts an alignment of the predicted amino acid sequences of mouse and human TLT-1 (SEQ ID NOs:2 and 5, respectively). The leader sequence is shown in bold, the cysteines forming disulfide bonds within the Ig V-type domain are boxed with dotted lines, potential O-glycosylation sites are marked by “Ŝ” for serine or “\check{T}” for threonine, the transmembrane domain is underlined, polyproline-rich region is boxed, and the ITIM sequence is boxed in gray. Asterisks indicates stop codons. Amino acid identities are indicated by dashes, while gaps are indicated by dots.

FIGS. 7A-7D depict TLT-1 expression in platelets. FIG. 7A: Northern analysis of mRNA isolated from mouse peripheral blood (lane 1) or bone marrow leukocytes (lane 2). Probes (TLT-1, TREM-1, and actin) are as indicated. FIG. 7B: Northern analysis of mRNA from macrophages ((Φ), platelets (P), PMN (N), monocytes (M), or unfractionated PBMC from human or mouse as indicated. Probes (TLT-1, TREM-1, and actin) were as indicated. FIG. 7C: Western blot analysis of lysates from HEK293T cells transfected as indicated immunoblotted with anti-TLT-1. FIG. 7D: Whole cell lysates from murine PBL, PBL cleared of platelets (PBL-PLT), bone marrow leukocytes (BM) or enriched platelets (PLT) were immunoblotted with anti-TLT-1 (top) followed by anti-actin (bottom).

FIGS. 8A-8B depict the regulation of surface TLT-1 by thrombin. FIG. 8A: Mouse platelets were stained with Anti-CD41 or Anti-CD62P as indicated and analyzed by FACS. FIG. 8B: Resting (left panels) or thrombin stimulated (right panels) platelets were stained Anti-CD62P and Anti-TLT-1 as indicated and analyzed by FACS.

FIG. 9 depicts the localization of TLT-1 to platelet and MK α-granules. Resting (top row) or thrombin stimulated (middle row) platelets,or in vitro derived MK (bottom row), were permeablized then stained with anti-TLT-1 (left column) and anti-CD62P (middle column) followed by a combination of Alexa 488 conjugated anti-rabbit and Alexa 633 conjugated anti-goat antibodies. The right column is an overlay of the left and middle images.

FIGS. 10A-10D depict the expression of TLT-1 in spleenic MKs and platelets. Frozen serial sections of murine spleen were fixed and stained with hematoxylin and eosin (FIG. 10A) or anti-TLT-1 followed by horseradish peroxidase conjugated anti-rabbit antibody (FIG. 10B), or secondary antibody alone (FIG. 10C). The approximate area demarcated in FIG. 10B is shown enlarged in FIG. 10D. Magnification of FIGS. 10A-10C is 200×. MKs are marked by arrows.

FIG. 11 depicts the nucleotide sequence of the splice variant of mouse TLT-1 isolated from RAW 264.7 cells (SEQ ID NO:18).

FIG. 12 depicts the amino acid sequence of the splice variant of mouse TLT-1 isolated from RAW 264.7 cells (SEQ ID NO:19).

FIG. 13 depicts the nucleotide sequence of the second splice variant of mouse TLT-1 (SEQ ID NO:21).

FIG. 14 depicts the amino acid sequence of the second splice variant of mouse TLT-1 (SEQ ID NO:22).

FIG. 15 depicts the nucleotide sequence of the splice variant of human TLT-1 (SEQ ID NO:24).

FIG. 16 depicts the amino acid sequence of the splice variant of human TLT-1 (SEQ ID NO:25).

FIG. 17 depicts a schematic of the mouse TLT-1 genomic rgion.

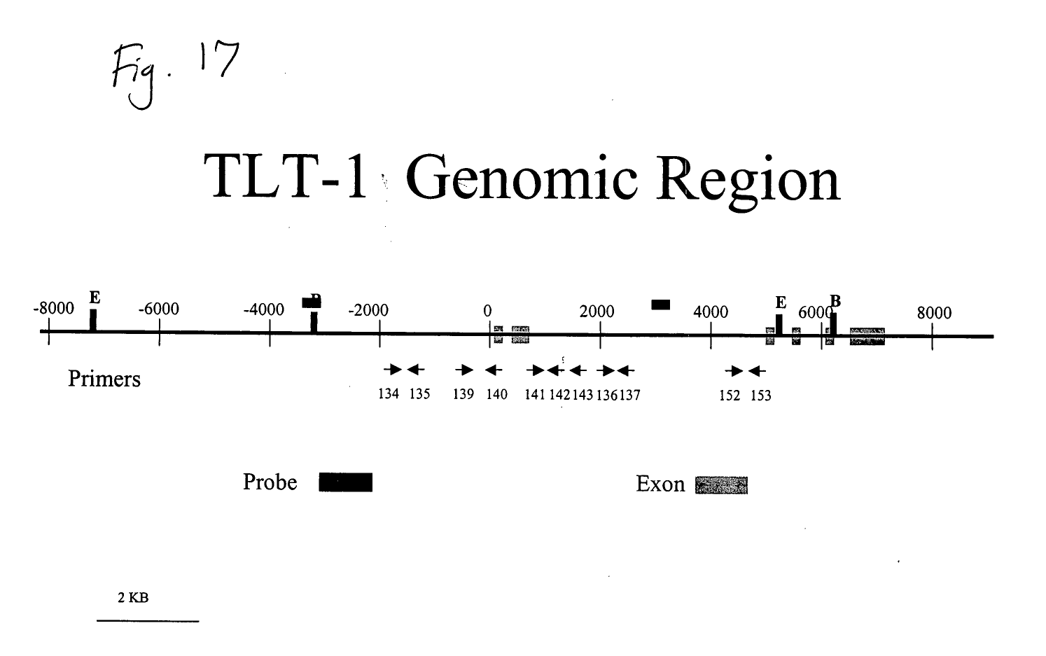

FIG. 18 depicts a schematic of TLT-1 knockout construct used to knock out the mouse TLT-1 and insert CFP (“knock-in”).

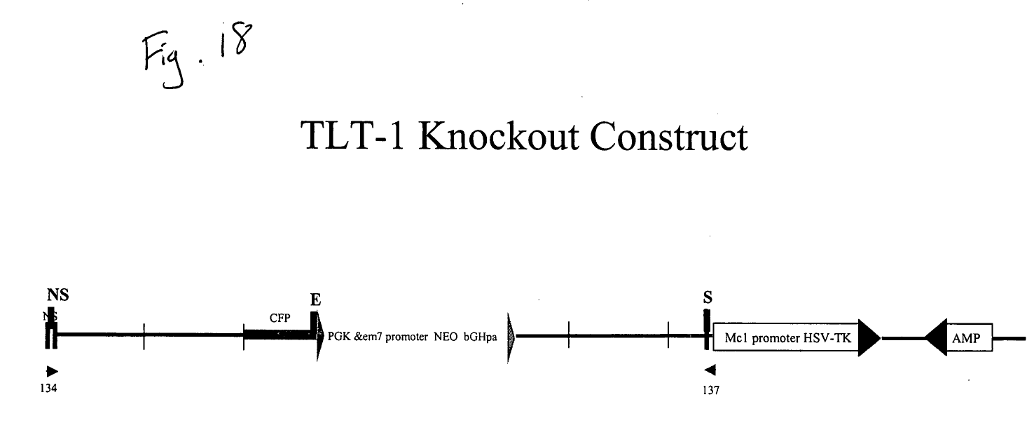

FIG. 19 depicts a schematic of the results of homogous recombination to make the mouse TLT-1 knockout/CFP knock-in.

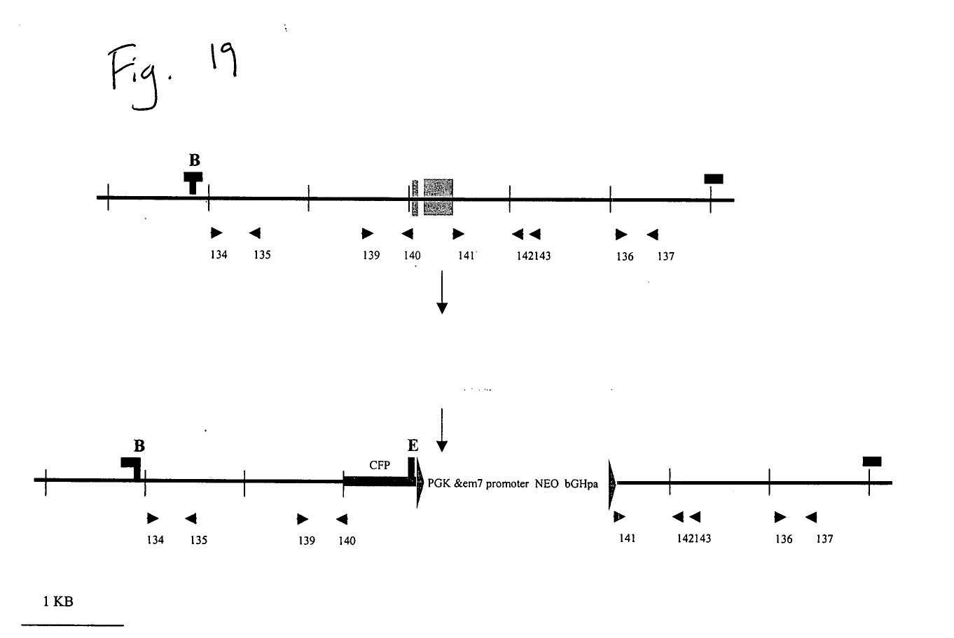

### DETAILED DESCRIPTION OF THE INVENTION

The present invention is based, at least in part, on the discovery of a novel inhibitory receptor within the TREM locus, referred to herein as TLT-1 (TREM-like transcript-1) nucleic acid and protein molecules. These novel molecules are capable of modulating platelet function and, thus, play a role in or function in a variety of cellular processes, e.g., blood clotting and/or immune function. The TLT-1 molecules of the present invention provide novel diagnostic targets and therapeutic agents to control immune disorders and platelet-associated disorders.

TLT-1 is the only inhibitory receptor described within a cluster of receptors known as the TREMs (Triggering receptors expressed on myeloid cells). TREMs 1 and 2 are known to modulate both innate and adaptive immunity. Specifically, TREM 1 amplifies the response to sepsis by inducing activation of neutrophils and other leukocytes. TREM 2 is reported to potentiate dentritic cell maturation. Therefore, TLT-1 may be important both in modulating septic shock and dendritic cell maturation and/or function.

TLT-1 is also highly expressed in peripheral blood platelets, and accordingly may be useful in modulating platelet function and in treating platelet-associated disorders.

As used herein, a “platelet-associated disorder” includes a disorder, disease or condition which is caused, characterized by, related to, or associated with a misregulation (e.g., downregulation or upregulation) of platelet activity. Platelet associated disorders also include disorders, diseases, or conditions which can be improved and/or treated by modulation of platelet activity. Platelet-associated disorders can detrimentally affect cellular functions such as blood-clotting, as well as other functions such as cellular proliferation, growth, differentiation, or migration, inter- or intra-cellular communication, tissue function, and systemic responses in an organism, such as immune responses. Preferred examples of platelet-associated disorders include, but are not limited to, immune disorders, septic shock, cancer (e.g., leukemias such as acute megakaryocytic leukemia, megakaryoblastic leukemia), infectious disease, stroke, heart disease, myocardial infarction, vascular disorders, arteriosclerosis, clotting and/or bleeding disorders, platelet insufficiency, and TLT-1 associated disorders.

Further examples of clotting and/or bleeding disorders include, but are not limited to, Hemophilia A (Factor VIII deficiency), Hemophilia B (Factor IX deficiency), von Willebrand disease, β-thalassemia, deep-vein thrombosis, thrombocytopenia, Immune Thrombocytopenic Purpura, Idiopathic Thrombocytopenic Purpura, Thrombotic Thrombocytopenic Purpura, hypercoagulation, hypocoagulation, protein S deficiency, protein C deficiency, Factor V Leiden, thrombosis, superficial vein thrombosis, phlebitis, thrombophlebitis, Factor XI deficiency (Rosenthal Syndrome or Plasma Thromboplastin Antecedent (PTA) deficiency), Factor XII deficiency (Hageman factor deficiency), Vitamin K deficiency, generalized coagulopathy, Factor XIII deficiency, Factor VII deficiency, internal bleeding, gastrointestinal bleeding, intracranial bleeding, pulmonary embolism, Afibrinogenemia, Dysfibrinogenemia, Factor II disorders, Factor III (tissue factor) associated disorders, Factor V (labile factor) deficiency, Factor X deficiency, Factor V & VIII Combined Deficiency, Factor VIII & IX combined Deficiency, Factor IX & XI Combined Deficiency, Thrombophilia (Antithrombin III deficiency), Giant Platelet Syndrome (platelet glycoprotein Ib deficiency), Fletcher Factor Deficiency (Prekallikrein deficiency), Autosomal dominant macrothrombocytopenia, the May-Hegglin anomaly, Sebastian syndrome, Fechtner syndrome, platelet storage pool deficiency, Chediak-Higashi syndrome, amegakaryocytic thrombocytopenia, thrombocytopenia with absent radii (TAR), radioulnar stenosis, familial platelet disorder with predisposition to acute myelocytic leukemia (FPD-AML), Platelet dense granule storage pool deficiency, grey platelet syndrome (also referred to as alpha granule deficiency), αδ-storage pool deficiency, Bernard-Soulier Syndrome, Glanzmann Thrombasthenia, Scott Syndrome, Alport Syndrome, Quebec Syndrome, White Sydrome, and Wiskott-Aldrich Syndrome; platelet-associated disorders caused or affected by common drugs, including, but not limited to, aspirin (ASA), non-steroidal anti-inflammatory drugs (e.g., indomethacin, ibuprofen and naproxen), ticlopidine, antibiotics, heart drugs, blood thinners, antidepressants, anaesthetics, and antihistamines; and clotting and/or bleeding disorders or conditions associated with surgery, organ transplants, bone marrow transplants, chronic kidney disease, chemotherapy, and/or other medical procedures and/or treatments.

In another embodiment, platelet-associated disorders include TLT-1-associated disorders, i.e., disorders, diseases or conditions which are caused, characterized by, related to, or associated with a misregulation (e.g., downregulation or upregulation) of TLT-1 expression and/or activity in any cell or tissue type in which TLT-1 may be expressed. Platelet-associated disorders can further detrimentally affect platelet-associated functions such as adhesion (e.g., via cell-cell and/or cell-matrix and/or basement membrane interactions), aggregation, secretion, procoagulant activity, and/or overall platelet number.

As used herein a “platelet”, also referred to as a “thrombocyte”, are nucleus-free cytoplasmic fragments derived from large cells in the bone marrow, the megakaryocyte. The central portion of a platelet stains purple with Wright's stain and is referred to as the granulomere. The peripheral portion stains clear and is called the hyalomere. Normal platelet counts range from 150,000 to 400,000 per cu/ml blood. Platelets play a crucial part in the blood clotting process by forming a platelet plug. This is a two step process. First, single platelets bind to the site of the wound (adhesion). Next, the platelets bind to each other (activation). Activation can be stimulated by components released when the blood vessel is damaged and by thrombin, released during the blood clotting process. When platelets become activated they change. They release agents which recruit and activate the surrounding platelets. The result of these two processes is the formation of fibrin which stabilizes the platelet plug, stops bleeding and allows injuries to heal.

As used herein, a “platelet-mediated activity” includes an activity which involves the action of platelets. Platelet-mediated activities include adhesion to the site of a wound, activation (e.g., release of blood clotting factors), induction of blood clotting (e.g., induction of fibrin formation), inhibition of bleeding, and induction of wound healing.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

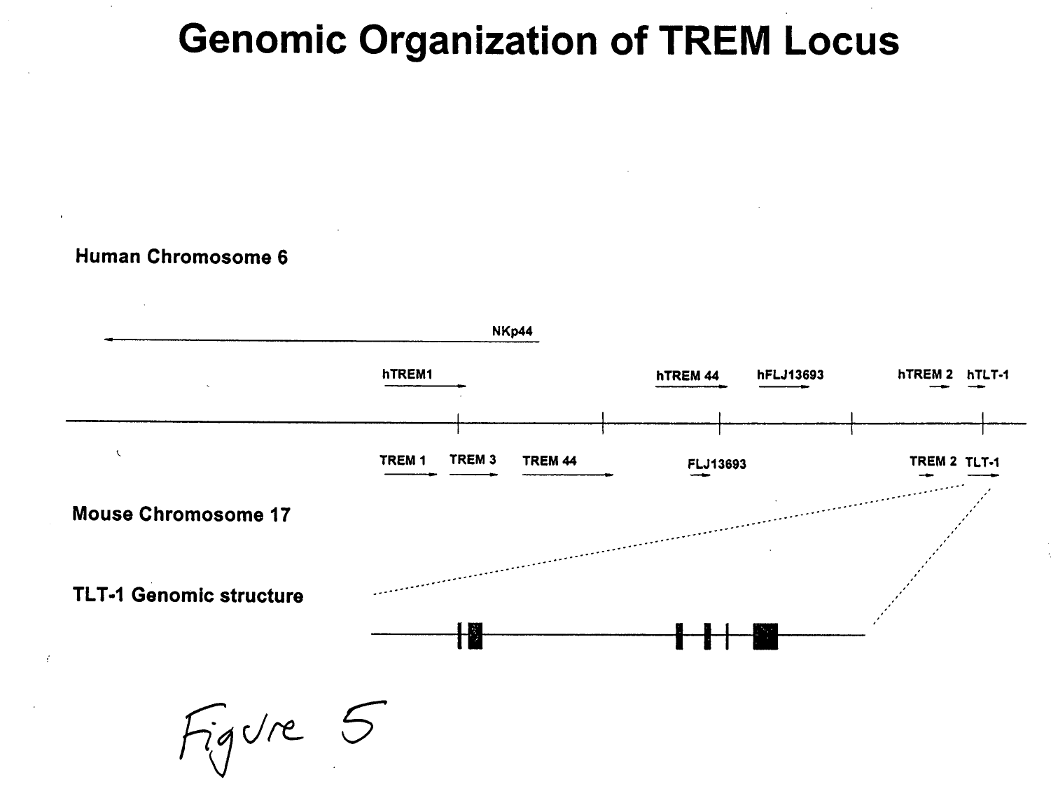

For example, the family of TLT-1 proteins comprises at least one “transmembrane domain”. As used herein, the term “transmembrane domain” includes an amino acid sequence of about 15, 20, 25, 30, 35, 40, or 45 amino acid residues in length which spans the plasma membrane. More preferably, a transmembrane domain includes about 23 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Ann. Rev. Neurosci. 19:235-263, the contents of which are incorporated herein by reference. Amino acid residues 181-203 of the native mouse TLT-1 protein (SEQ ID NO:2) and amino acid residues 165-187 of the mature mouse TLT-1 protein are predicted to comprise a transmembrane domain (see FIG. 5). Amino acid residues 164-186 of the native human TLT-1 protein (SEQ ID NO:5) and residues 149-171 of the mature human TLT-1 protein are predicted to comprise a transmembrane domain (see FIG. 5).

In another embodiment of the invention, a TLT-1 protein of the present invention is identified based on the presence of a signal peptide. The prediction of such a signal peptide can be made, for example, utilizing the computer algorithm SignalP (Henrik et al. (1997) Prot. Eng. 10:1-6). As used herein, a “signal sequence”, “signal peptide”, or “leader sequence” includes a peptide containing about 15 or more amino acids which occurs at the N-terminus of secretory and membrane bound proteins and which contains a large number of hydrophobic amino acid residues. For example, a signal sequence contains at least about 10-30 amino acid residues, preferably about 12-25 amino acid residues more preferably about 14-20 amino acid residues, and most preferably about 15 or 16 amino acid residues, and has at least about 35-65%, preferably about 38-50%, and more preferably about 40-45% hydrophobic amino acid residues (e.g., Valine, Leucine, Isoleucine or Phenylalanine). Such a “signal sequence”, also referred to in the art as a “signal peptide” or “leader sequence”, serves to direct a protein containing such a sequence to a lipid bilayer, and is cleaved in secreted and membrane bound proteins. A signal sequence was identified in the amino acid sequence of mouse TLT-1 at about amino acids 1-16 of SEQ ID NO:2 (FIG. 5). A signal sequence was also identified in the amino acid sequence of human TLT-1 at about amino acids 1-15 of SEQ ID NO:5 (FIG. 5).

As used herein the term “mature polypeptide” refers to a polypeptide, e.g., a TLT-1 polypeptide of the present invention, in which the signal peptide has been removed (i.e., cleaved off), as contrasted with a “native polypeptide”, which refers to a polypeptide, e.g., a TLT-1 polypeptide of the present invention (e.g., SEQ ID NO:2 or SEQ ID NO:5), in which the signal peptide is intact. As used herein, the term “mature mouse TLT-1 polypeptide” includes a polypeptide comprising amino acid residues 17-322 of SEQ ID NO:2. When referring to the amino acid sequence of the mature mouse TLT-1 polypeptide, it will be understood by those of skill in the art that amino acid residues 1-306 of the mature polypeptide correspond, respectively, to amino acid residues 17-322 of SEQ ID NO:2. As used herein, the term “mature human TLT-1 polypeptide” includes a polypeptide comprising amino acid residues 16-311 of SEQ ID NO:5. When referring to the amino acid sequence of the mature human TLT-1 polypeptide, it will be understood by those of skill in the art that amino acid residues 1-296 of the mature polypeptide correspond, respectively, to amino acid residues 16-311 of SEQ ID NO:5.

In another embodiment, a TLT-1 molecule of the present invention is identified based on the presence of an “IgV domain” (also referred to as an “Ig V-type domain”) in the polypeptide or corresponding nucleic acid molecule. As used herein, IgV domains and the related IgC domains are recognized in the art as Ig superfamily member domains. These domains correspond to structural units that have distinct folding patterns called Ig folds. Ig folds are comprised of a sandwich of two β sheets, each consisting of antiparallel β strands of about 5-10 amino acids with a conserved disulfide bond between the two sheets in most, but not all, domains. IgC domains of Ig, TCR, and MHC molecules share the same types of sequence patterns and are called the C1 set within the Ig superfamily. Other IgC domains fill within other sets. IgV domains also share sequence patterns and are called V set domains. IgV domains are longer than C-domains and form an additional pair of β strands. In a preferred embodiment, an IgV domain in the TLT-1 molecules of the present invention comprises at least two cysteine residues, between which can form a disulfide bond. In another preferred embodiment, an IgV domain of the present invention is encoded by exon 2 the TLT-1 gene. In a further preferred embodiment, an IgV domain of the TLT-1 molecules of the present invention comprises about 50-150 amino acid residues more preferably about 60-140, 70-130, 80-120, 90-110, or most preferably about 101 amino acid residues. Amino acid residues 26-126 of the native mouse TLT-1 polypeptide (SEQ ID NO:2), and amino acid residues 10-110 of the predicted mature mouse polypeptide, are predicted to comprise an IgV domain. The predicted IgV domain identified in the mouse TLT-1 polypeptide comprises two cysteine residues predicted to form a disulfide bond; these two cysteine residues are located at residues 39 and 105 of the native mouse TLT-1 polypeptide (SEQ ID NO:2) and at residues 23 and 89 of the predicted mature mouse polypeptide Amino acid residues 25-125 of the native human TLT-1 polypeptide (SEQ ID NO:5), and amino acid residues 10-110 of the predicted mature human polypeptide, are predicted to comprise an IgV domain. The predicted IgV domain identified in the human TLT-1 polypeptide comprises two cysteine residues predicted to form a disulfide bond; these two cysteine residues are located at residues 38 and 104 of the native human TLT-1 polypeptide (SEQ ID NO:5) and at residues 23 and 89 of the predicted mature human polypeptide.

In another embodiment, a TLT-1 molecule of the present invention is identified based on the presence of a “extracellular domain” in the polypeptide or corresponding nucleic acid molecule. As used herein, the term “extracellular domain” represents the N-terminal amino acids which extend as a tail from the surface of a cell. In a preferred embodiment, an extracellular domain comprises about 100-230, 110-220, 120-210, 130-200, 140-190, or most preferably, about 148, 163, 164, or 180 amino acid residues. Preferably, an extracellular domain of the present invention includes an IgV domain, at least two cysteine residues which can form a disulfide bond, and at least one or two potential O-glycosylation sites, and may include a signal peptide domain. Amino acid residues 1-180 of the native mouse TLT-1 polypeptide (SEQ ID NO:2), and amino acid residues 1-164 of the predicted mature mouse polypeptide, are predicted to comprise an extracellular domain. Amino acid residues 1-163 of the native human TLT-1 polypeptide (SEQ ID NO:5), and amino acid residues 1-148 of the predicted mature human polypeptide, are also predicted to comprise an extracellular domain.

In still another embodiment, a TLT-1 molecule of the present invention is identified based on the presence of a “cytoplasmic domain” in the polypeptide or corresponding nucleic acid molecule. As used herein, the term “cytoplasmic domain”, also referred to herein as an “intracellular domain”, represents the C-terminal amino acids which extend as a tail into the cytoplasm of a cell. In a preferred embodiment a cytoplasmic domain comprises about 50-200, 70-180, 90-160, 110-140, or most preferably, about 119 or 127 amino acid residues. Preferably, a cytoplasmic domain of the present invention comprises at least one polyproline-rich region, one PEST domain, and/or one ITIM, as described elsewhere herein. Amino acid residues 204-322 of the native mouse TLT-1 polypeptide (SEQ ID NO:2), and amino acid residues 188-306 of the predicted mature mouse polypeptide, are predicted to comprise a cytoplasmic domain. Amino acid residues 187-311 of the native human TLT-1 polypeptide (SEQ ID NO:5), and amino acid residues 172-296 of the predicted mature human polypeptide, are predicted to comprise cytoplasmic domains.

In another embodiment, a TLT-1 molecule of the present invention is identified based on the presence of a polyproline-rich region. As used herein the term “polyproline rich region”, also referred to herein as a “polyproline-rich segment”, includes a domain or motif rich in proline residues, which mediates binding to SH3 domains and/or WW domains, which may be found in other proteins, e.g., TLT-1 target molecules. Preferably, a polyproline-rich region comprises about 7-19, 8-18, 9-17, 10-16, 11-15, or most preferably about 12 or 14 amino acid residues, roughly about half of which are proline residues. Preferably, a polyproline-rich region is found within a cytoplasmic domain. A polyproline-rich region was identified at about residues 269-280 of the native mouse TLT-1 polypeptide (SEQ ID NO:2), and at about residues 253-264 of the predicted mature mouse polypeptide. A polyproline-rich region was identified at about residues 258-271 of the native human TLT-1 polypeptide (SEQ ID NO:5), and at about residues 243-256 of the predicted mature human polypeptide.

In still another embodiment a TLT-1 molecule of the present invention is identified based on the presence oft an ITIM. As used herein, the term “ITIM”, also referred to as an “immunoreceptor tyrosine-based inhibitory motif”, includes a motif which mediates inhibition through the recruitment of SH2-domain containing protein tyrosine phosphatases (e.g., SHP-1) and/or lipid phosphatases (e.g., SHIP-1). Preferably, an ITIM is found within a cytoplasmic domain. ITIMs are further described in Sinclair, N. R. (2000) Crit. Rev. Immunol. 20(2):89-102, as well as in Burshtyn, D. N. et al. (1997) J. Biol. Chem. 272(20):13066-72. An ITIM was identified at about residues 289-295 of the native mouse TLT-1 polypeptide (SEQ ID NO:2) and at about residues 273-279 of the predicted mature mouse polypeptide. An ITIM was identified at about residues 280-286 of the native human TFT-1 polypeptide (SEQ ID NO:5) and at about residues 265-271 of the predicted mature human TLT-1 polypeptide. A second ITIM was identified at about residues 244-249 of the native human TLT-1 polypeptide (SEQ ID NO:5) and at about residues 229-234 of the mature human TLT-1 polypeptide. Notably, this second ITIM contains a threonine residue (T) at the −2 position.

In another embodiment, a TLT-1 molecule of the present invention is identified based on the presence of at least one PEST sequence. As used herein, a PEST sequence is a polypeptide sequences enriched in proline (P), glutamic acid (E), serine (S) and threonine (T) that may target the protein for rapid degradation (Rechsteinera, M. and Rogers, S. W. (1996) Trends Biochem. Sci. 21:267-271.). Preferably, a PEST sequence in the TLT-1 molecules of present invention comprises about 10-60 amino acid residues and scores at least about 5 on the scale disclosed by Rechsteiner and Rogers. More preferably, a PEST sequence in the TLT-1 molecules of the present invention comprises about 15-55, 20-50, 25-45, or 30-40 amino acid residues and scores at least about 7, 9, 11, or 13 on the scale disclosed by Rechsteiner and Rogers. Most preferably, a PEST sequence in the TLT-1 molecules of the present invention comprises about 35 or 38 amino acid residues and scores at least about 14.25 on the scale disclosed by Rechsteiner and Rogers. A PEST sequence was identified at about residues 246-280 of the native mouse TLT-1 polypeptide (SEQ ID NO:2) and at about residues 230-264 of the mature mouse TLT-1 polypeptide. A PEST sequence was identified at about residues 234-271 of the native human TLT-1 polypeptide (SEQ ID NO:5) and at about residues 219-256 of the mature human TLT-1 polypeptide.

In still another embodiment, a TLT-1 molecule of the present invention is identified based on the presence of at least one potential O-glycosylation site, preferably two potential O-glycosylation sites. Two potential O-glycosylation sites were identified in the amino acid sequence of the mouse TLT-1 polypeptide, at about residues 34 (serine) and 65 (threonine) of SEQ ID NO:2. Two potential O-glycosylation sites were identified in the amino acid sequence of the human TLT-1 polypeptide, at about residues 33 and 64 of SEQ ID NO:5.

In a preferred embodiment the TLT-1 molecules of the invention include at least one or more of the following domains: a transmembrane domain, a signal peptide domain, and IgV domain, a cysteine residue, an extracellular domain, a cytoplasmic domain, a polyproline-rich region, an ITIM a PEST domain, and an O-glycosylation site.

Isolated proteins of the present invention, preferably TLT-1 proteins, have an amino acid sequence sufficiently identical to the amino acid sequence of SEQ ID NO:2 or 5, or are encoded by a nucleotide sequence sufficiently identical to SEQ ID NO:1, 3, 4, or 6. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, a “TLT-1 activity”, “biological activity of TLT-1” or “functional activity of TLT-1”, refers to an activity exerted by a TLT-1 protein, polypeptide or nucleic acid molecule on a TLT-1 responsive cell or tissue, or on a TLT-1 protein target molecule, as determined in vivo, or in vitro, according to standard techniques. In one embodiment, a TLT-1 activity is a direct activity, such as an association with a TLT-1-target molecule. As used herein, a “target molecule” or “binding partner” is a molecule with which a TLT-1 protein binds or interacts in nature, such that TLT-1-mediated function is achieved. A TLT-1 target molecule can be a non-TLT-1 molecule or a TLT-1 protein or polypeptide of the present invention. In an exemplary embodiment, a TLT-1 target molecule is a second TLT-1 molecule, a TLT-1 ligand, Src, SHP-1, or SHIP-1. Alternatively, a TLT-1 activity is an indirect activity, such as a cellular signaling activity mediated by interaction of the TLT-1 protein with a TLT-1 ligand.

The biological activities of TLT-1 are described herein. For example, the TLT-1 proteins of the present invention can have one or more of the following activities: 1) interaction with a TLT-1 target molecule (e.g., a second TLT-1 molecule, or a non-TLT-1 molecule such as a TLT-1 specific antibody, a TLT-1 ligand, a cell-surface protein, a Src family member SHP-1, SHP-2, SHIP-1, an SH2 domain containing protein, an SH3 domain containing protein, and/or a WW domain containing protein); 2) modulation of megakaryocyte differentiation; 3) modulation of platelet differentiation and/or production (thrombopoiesis); 4) modulation of platelet activity; 5) modulation of intra- or inter-cellular signaling; 6) localization to platelet and/or megakaryocyte alpha granules: 7) modulation of platelet and/or megakaryocyte granule formation and/or sorting; 8) localization to the platelet and/or megakaryocyte cell surface; 9) modulation of platelet interaction with and/or adhesion to the extracellular matrix and/or basement membrane; 10) modulation of blood clotting; 11) modulation of bleeding; 12) modulation of immune responses; 13) modulation of activation of neutrophils and/or other leukocytes; 14) modulation of dendritic cell maturation and/or function; and/or 15) modulation of cellular proliferation.

Accordingly, another embodiment of the invention features isolated TLT-1 proteins and polypeptides having a TLT-1 activity. Other preferred proteins are TLT-1 proteins having one or more of the following domains: a transmembrane domain, a signal peptide domain and IgV domain, a cysteine residue, all extracellular domain, a cytoplasmic domain, a polyproline-rich region, an ITIM, a PEST domain, and an O-glycosylation site and, preferably, a TLT-1 activity.

Additional preferred proteins have at least one a transmembrane domain, a signal peptide domain, and IgV domain, a cysteine residue, an extracellular domain, a cytoplasmic domain, a polyproline-rich region, an ITIM, a PEST domain, and an O-glycosylation site, and are, preferably, encoded by a nucleic acid molecule having a nucleotide sequence which hybridizes under stringent hybridization conditions to a complement of a nucleic acid molecule comprising the nucleotide sequence of SEQ ID NO:1, 3, 4, 6, 18, 20, 21, 23, 24, or 26.

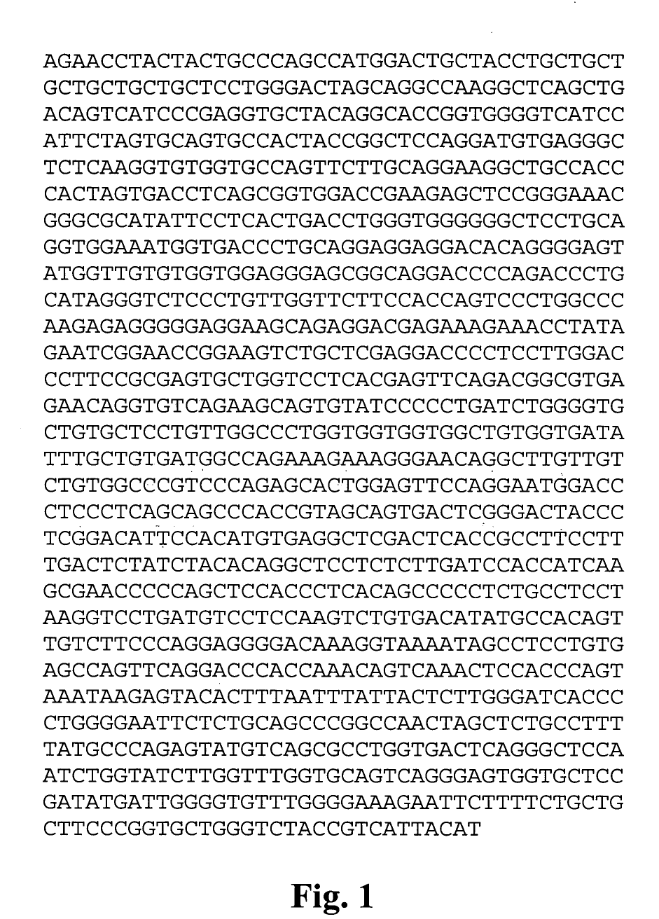

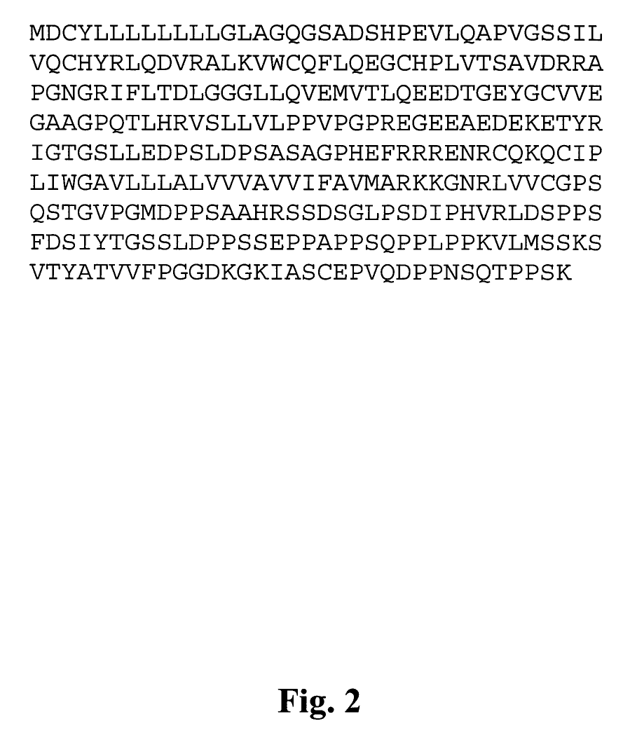

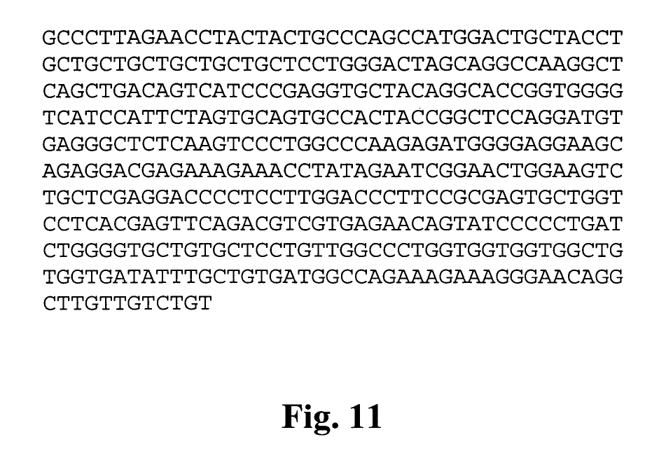

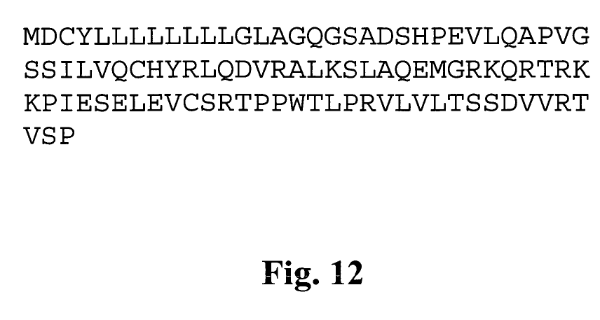

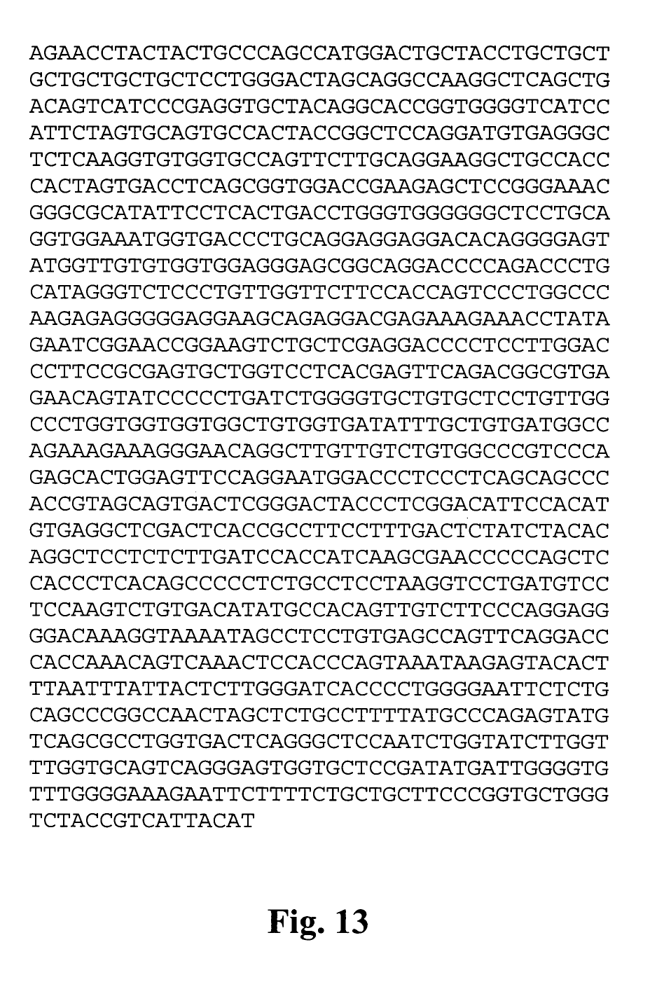

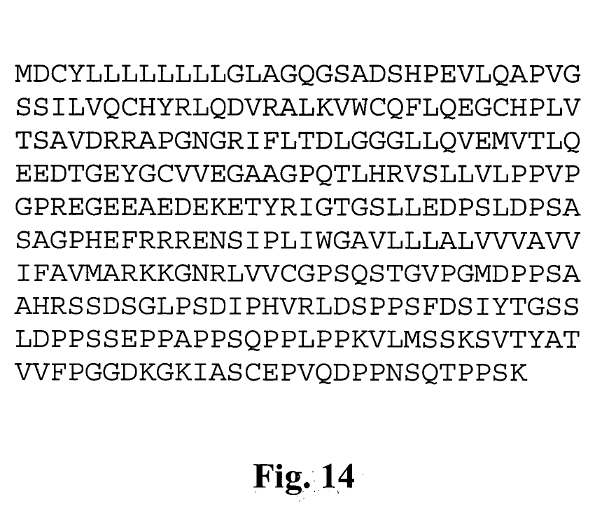

The nucleotide sequence of the isolated mouse TLT-1 cDNA and the predicted amino acid sequence of the mouse TLT-1 polypeptide are shown in FIGS. 1 and 2, respectively, and in SEQ ID NOs:1 and 2, respectively. The coding sequence of mouse TLT-1 is set forth as SEQ ID NO:3. The nucleotide sequence of a splice variant of mouse TLT-1 isolated from RAW 264.7 cells and the predicted amino acid sequence of the polypeptide encoded by that splice variant are shown in FIGS. 11 and 12, respectively, and in SEQ ID NOs:18 and 19, respectively. The coding sequence of the RAW 264.7 splice variant is set forth as SEQ ID NO:20. The nucleotide sequence of a second splice variant of mouse TLT-1 and the predicted amino acid sequence of the polypeptide encoded by that splice variant are shown in FIGS. 13 and 14, respectively, and in SEQ ID NOs:21 and 22, respectively. The coding sequence of the second splice variant of mouse TLT-1 is set forth as SEQ ID NO:23.

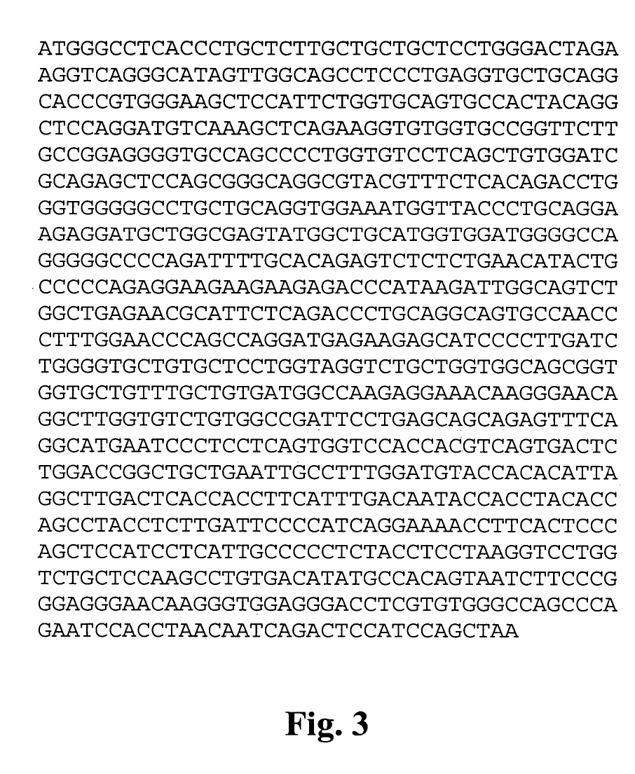

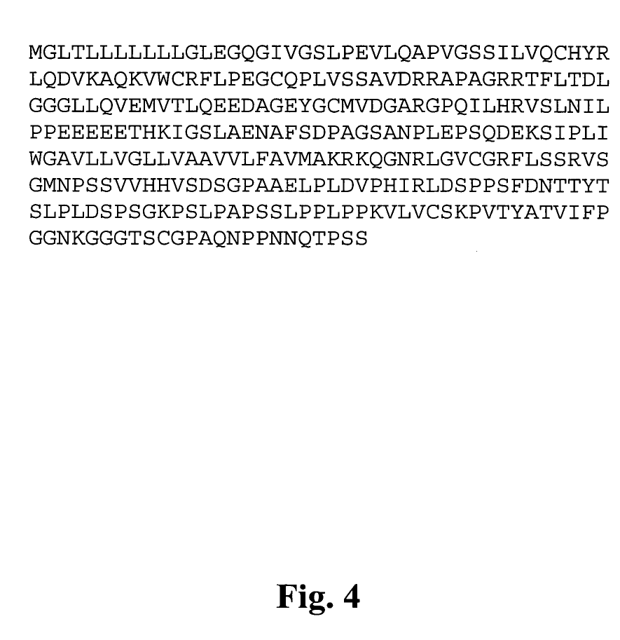

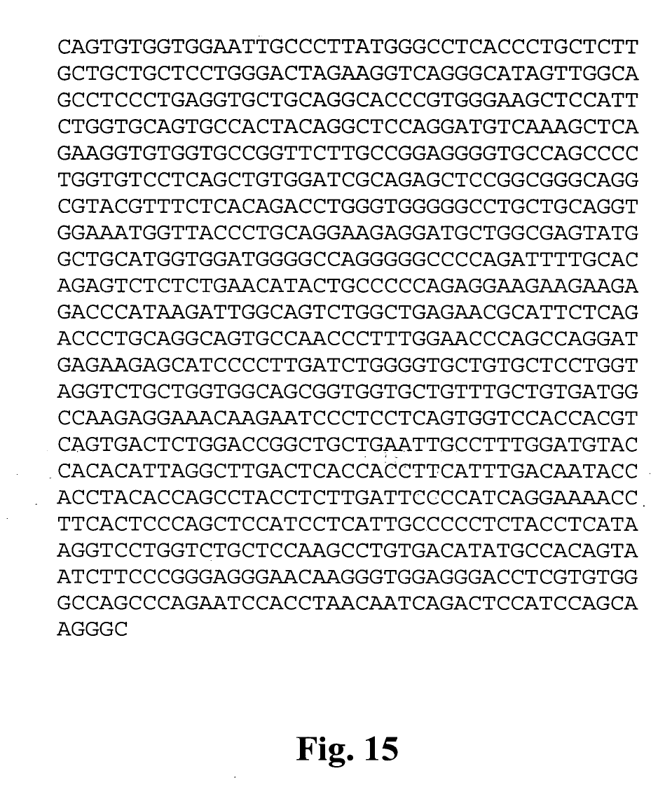

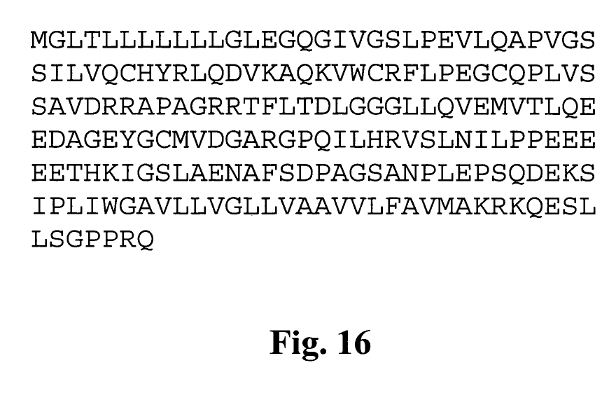

The nucleotide sequence of the isolated human TLT-1 cDNA and the predicted amino acid sequence of the human TLT-1 polypeptide are shown in FIGS. 3 and 4, respectively, and in SEQ ID NOs:4 and 5, respectively. The coding sequence of human TLT-1 is set forth as SEQ ID NO:6. The nucleotide sequence of a splice variant of human TLT-1 and the predicted amino acid sequence of the polypeptide encoded by that splice variant are shown in FIGS. 15 and 16, respectively, and in SEQ ID NOs:24 and 25, respectively. The coding sequence of the splice variant of human TLT-1 is set forth as SEQ ID NO:26.

The mouse TLT-1 gene, which is approximately 1220 nucleotides in length, encodes a protein having a molecular weight of approximately 36 kD and which is approximately 322 amino acid residues in length. The mouse RAW 264.7 splice variant, which is approximately 422 nucleotides in length, encodes a protein having a molecular weight of approximately 11 kD and which is approximately 99 amino acid residues in length. The second splice variant of mouse TLT-1, which is approximately 1205 nucleotides in length, encodes a pteoin having a molecular weight of approximately 35 kD and which is approximately 317 amino acid residues in length.

The human TLT-1 gene, which is approximately 936 nucleotides in length, encodes a protein having a molecular weight of approximately 34 kD and which is approximately 311 amino acid residues in length. The splice variant of human TLT-1, which is approximately 907 nucleotides in length, encodes a protein having a molecular weight of approximately 22 kD and which is approximately 199 amino acid residues in length.

Various aspects of the invention are described in further detail in the following subsections:

I. Isolated Nucleic Acid Molecules

One aspect of the invention pertains to isolated nucleic acid molecules that encode TLT-1 proteins or biologically active portions thereof, as well as nucleic acid fragments sufficient for use as hybridization probes to identify TLT-1-encoding nucleic acid molecules (e.g., TLT-1 mRNA) and fragments for use as PCR primers for the amplification or mutation of TLT-1 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TLT-1 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 3, 4, 6, 18, 20, 21, 23, 24, or 26, or a portion or complement thereof, can be isolated using standard molecular biology techniques and the sequence information provided herein. Using all or portion of the nucleic acid sequence of SEQ ID NO:1, 3, 4, 6, 18, 20, 21, 23, 24, or 26 as a hybridization probe, TLT-1 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 4, 6, 18, 20, 21, 23, 24, or 26 can be isolated by the polymerase chain reaction (PCR) using synthetic oligonucleotide primers designed based upon the sequence of SEQ ID NO:1, 3, 4, 6, 18, 20, 21, 23, 24, or 26.

A nucleic acid of the invention can be amplified using cDNA, mRNA or, alternatively, genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to TLT-1 nucleotide sequences can be prepared by standard synthetic techniques, e.g., using an automated DNA synthesizer.

In a preferred embodiment, an isolated nucleic acid molecule of the invention comprises the nucleotide sequence shown in SEQ NO:1, 3, 4, 6, 18, 20, 21, 23, 24, or 26. This cDNA may comprise sequences encoding the mouse TLT-1 protein (i.e., “the coding region”, from nucleotides 22-987, corresponding to SEQ ID NO:3), as well as 5′ untranslated sequences (nucleotides 1-21) and 3′ untranslated sequences (nucleotides 988-1220) of SEQ ID NO:1. This cDNA may also comprise sequences encoding the mouse TLT-1 protein (i.e., “the coding region”, from nucleotides 22-987), as well as a stop codon (e.g., nucleotides 988-990 of SEQ ID NO:1). Alternatively, the nucleic acid molecule can comprise only the coding region of SEQ ID NO:1 (e.g., nucleotides 22-987, corresponding to SEQ ID NO:3).

This cDNA may also comprise sequences encoding the mouse RAW 264.7 splice variant protein (i.e., “the coding region”, from nucleotides 28-324, corresponding to SEQ ID NO:20), as well as 5′ untranslated sequences (nucleotides 1-27) and 3′ untranslated sequences (nucleotides 325-422) of SEQ ID NO:18. This cDNA may also comprise sequences encoding the mouse RAW 264.7 splice variant protein (i.e., “the coding region”, from nucleotides 28-324, corresponding to SEQ ID NO:20), as well as a stop codon (e.g., nucleotides 325-327 of SEQ ID NO:21). Alternatively, the nucleic acid molecule can comprise only the coding region of SEQ ID NO:18 (e.g., nucleotides 28-324, corresponding to SEQ ID NO:20).

This cDNA may also comprise sequences encoding the second mouse splice variant protein (i.e., “the coding region”, from nucleotides 22-972, corresponding to SEQ ID NO:23), as well as 5′ untranslated sequences (nucleotides 1-21) and 3′ untranslated sequences (nucleotides 973-1205) of SEQ ID NO:21. This cDNA may also comprise sequences encoding the encoding the second mouse splice variant protein (i.e., “the coding region”, from nucleotides 22-972, corresponding to SEQ ID NO:23), as well as a stop codon (e.g., nucleotides 973-975 of SEQ ID NO:21). Alternatively, the nucleic acid molecule can comprise only the coding region of SEQ ID NO:21 (e.g., nucleotides 22-972, corresponding to SEQ ID NO:23).

This cDNA may comprise sequences encoding the human TLT-1 protein (i.e., “the coding region”, from nucleotides 1-933 of SEQ ID NO:4, corresponding to SEQ ID NO:6). This cDNA may also comprise sequences encoding the human TLT-1 protein (i.e., “the coding region”, from nucleotides 1-933), as well as a stop codon (e.g., nucleotides 934-936 of SEQ ID NO:4). Alternatively, the nucleic acid molecule can comprise only the coding region of SEQ ID NO:4 (e.g., nucleotides 1-933, corresponding to SEQ ID NO:6).

This cDNA may also comprise sequences encoding the human splice variant protein (i.e., “the coding region”, from nucleotides 22-618, corresponding to SEQ ID NO:26), as well as 5′ untranslated sequences (nucleotides 1-21) and 3′ untranslated sequences (nucleotides 619-907) of SEQ ID NO:24. This cDNA may also comprise sequences encoding the encoding the human splice variant protein (i.e., “the coding region”, from nucleotides 22-618, corresponding to SEQ ID NO:26), as well as a stop codon (e.g., nucleotides 619-621 of SEQ ID NO:24). Alternatively, the nucleic acid molecule can comprise only the coding region of SEQ ID NO:24 (e.g., nucleotides 22-618, corresponding to SEQ ID NO:26).

In another preferred embodiment, an isolated nucleic acid molecule of the invention comprises a nucleic acid molecule which is a complement of the nucleotide sequence shown in SEQ ID NO:1, 3, 4, 6, 18, 20, 21, 23, 24, or 26, or a portion of any of these nucleotide sequences. A nucleic acid molecule which is complementary to the nucleotide sequence shown in SEQ ID NO:1, 3, 4, 6, 18, 20, 21, 23, 24, or 26, is one which is sufficiently complementary to the nucleotide sequence shown in SEQ ID NO:1, 3, 4, 6, 18, 20, 21, 23, 24, or 26 such that it can hybridize to the nucleotide sequence shown in SEQ ID NO:1, 3, 4, 6, 18, 20, 21, 23, 24, or 26, respectively, thereby forming a stable duplex.

In still another preferred embodiment, an isolated nucleic acid molecule of the present invention comprises a nucleotide sequence which is at least about 50%, 55%, 60%, 65%, 70%, 75%, 80%, 81%, 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to the entire length of the nucleotide sequence shown in SEQ ID NO:1, 3, 4, 6, 18, 20, 21, 23, 24, or 26, or a portion of any of these nucleotide sequences.

Moreover, the nucleic acid molecule of the invention can comprise only a portion of the nucleic acid sequence of SEQ ID NO:1, 3, 4, 6, 18, 20, 21, 23, 24, or 26, for example, a fragment which can be used as a probe or primer or a fragment encoding a portion of a TLT-1 protein, e.g., a biologically active portion of a TLT-1 protein. The nucleotide sequences determined from the cloning of the mouse and human TLT-1 genes allow for the generation of probes and primers designed for use in identifying and/or cloning other TLT-1 family members, as well as TLT-1 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 4, 6, 18, 20, 21, 23, 24, or 26, of an anti-sense sequence of SEQ ID NO:1, 3, 4, 6, 18, 20, 21, 23, 24, or 26, or of a naturally occurring allelic variant or mutant of SEQ. ID NO:1, 3, 4, 6, 18, 20, 21, 23, 24, or 26. In one embodiment, a nucleic acid molecule of the present invention comprises a nucleotide sequence which is greater than 10, 20, 30, 40, 50, 100, 150, 200, 250, 300, 333, 336, 350, 400, 450, 487, 500, 519, 550, 600, 650, 700, 750, 800, 850, 900, 950, 1000, 1050, 1100, 1150, 1200, 1210 or more nucleotides in length and hybridizes under stringent hybridization conditions to a nucleic acid molecule of SEQ ID NO:1, 3, 4, 6, 18, 20, 21, 23, 24, or 26.

Probes based on the TLT-1 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a TLT-1 protein, such as by measuring a level of a TLT-1-encoding nucleic acid in a sample of cells from a subject e.g., detecting TLT-1 mRNA levels or determining whether a genomic TLT-1 gene has been mutated or deleted.

Probes based on the TLT-1 nucleotide sequence can also be used in ribonuclease protection assays. In a preferred embodiment, kits can be provided that provide at least one, and preferably more than one, probes for use in ribonuclease protection assays.

A nucleic acid fragment encoding a “biologically active portion of a TLT-1 protein” can be prepared by isolating a portion of the nucleotide sequence of SEQ ID NO:1, 3, 4, 6, 18, 20, 21, 23, 24, or 26 which encodes a polypeptide having a TLT-1 biological activity (the biological activities of the TLT-1 proteins are described herein), expressing the encoded portion of the TLT-1 protein (e.g., by recombinant expression in vitro) and assessing the activity of the encoded portion of the TLT-1 protein.

The invention further encompasses nucleic acid molecules that differ from the nucleotide sequence shown in SEQ ID NO:1, 3, 4, 6, 18, 20, 21, 23, 24, or 26 due to degeneracy of the genetic code and thus encode the same TLT-1 proteins as those encoded by the nucleotide sequence shown in SEQ ID NO:1, 3, 4, 6, 18, 20, 21, 23, 24, or 26. In another embodiment, an isolated nucleic acid molecule of the invention has a nucleotide sequence encoding a protein having an amino acid sequence shown in SEQ ID NO:25, 19, 22, or 25.

In addition to the TLT-1 nucleotide sequences shown in SEQ ID NO:1, 3, 4, 6, 18, 20, 21, 23, 24, or 266, it will be appreciated by those skilled in the art that DNA sequence polymorphisms that lead to changes in the amino acid sequences of the TLT-1 proteins may exist within a population (e.g., the human or mouse population). Such genetic polymorphism in the TLT-1 genes may exist among individuals within a population due to natural allelic variation. As used herein, the terms “gene” and “recombinant gene” refer to nucleic acid molecules which include an open reading frame encoding a TLT-1 protein, preferably a mammalia TLT-1 protein, and can further include non-coding regulatory sequences, and introns.

Allelic variants of human TLT-1 include both functional and non-functional TLT-1 proteins. Functional allelic variants are naturally occurring amino acid sequence variants of the human TLT-1 protein that maintain the ability to bind a TLT-1 ligand or target molecule and/or modulate platelet activity. Functional allelic variants will typically contain only conservative substitution of one or more amino acids of SEQ ID NO:2, 5, 19, 22, or 25, or substitution, deletion or insertion of non-critical residues in non-critical regions of the protein.

Non-functional allelic variants are naturally occurring amino acid sequence variants of the human TLT-1 protein that do not have the ability to either bind a TLT-1 ligand and/or modulate any of the TLT-1 activities described herein. Non-functional allelic variants will typically contain a non-conservative substitution, a deletion, or insertion or premature truncation of the amino acid sequence of SEQ ID NO:2, 5, 19, 22, or 25, or a substitution, insertion or deletion in critical residues or critical regions of the protein.

The present invention further provides non-human orthologues of the human TLT-1 protein. Orthologues of the mouse or human TLT-1 proteins are proteins that are isolated from non-human organisms and possess the same TLT-1 ligand binding and/or modulation of platelet activities of the human TLT-1 protein. Orthologues of the mouse or human TLT-1 proteins can readily be identified as comprising an amino acid sequence that is substantially identical to SEQ ID NO:2, 5, 19, 22, or 25.

Moreover, nucleic acid molecules encoding other TLT-1 family members and, thus, which have a nucleotide sequence which differs from the TLT-1 sequences of SEQ ID NO:1, 3, 4, 6, 18, 20, 21, 23, 24, or 26 are intended to be within the scope of the invention. For example, another TLT-1 cDNA can be identified based on the nucleotide sequence of mouse or human TLT-1. Moreover, nucleic acid molecules encoding TLT-1 proteins from different species, and which, thus, have a nucleotide sequence which differs from the TLT-1 sequences of SEQ ID NO:1, 3, 4, 6, 18, 20, 21, 23, 24, or 26 are intended to be within the scope of the invention. For example, a rat or monkey TLT-1 cDNA can be identified based on the nucleotide sequence of a mouse or human TLT-1.

Nucleic acid molecules corresponding to natural allelic variants and homologues of the TLT-1 cDNAs of the invention can be isolated based on their homology to the TLT-1 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the TLT-1 cDNAs of the invention can further be isolated by mapping to the same chromosome or locus as the TLT-1 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, 3, 4, 6, 18, 20, 21, 23, 24, or 26. In other embodiment, the nucleic acid is at least 10, 20, 30, 40, 50, 100, 150, 200, 250, 300, 333, 336, 350, 400, 450, 487, 500, 519, 550, 600, 650, 700, 750, 800, 850, 900, 950, 1000, 1050, 1100, 1150, 1200, or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25mM EDTA, pH 7.4) can be substituted for SSC (1×SSC is 0.15M NaCl and 15mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule of the invention that hybridizes under stringent conditions to the sequence of SEQ ID NO:1, 3, 4, 6, 18, 20, 21, 23, 24, or 26, and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TLT-1 sequences that may exist in the population, the skilled artisan will further appreciate that changes can be introduced by mutation into the nucleotide sequences of SEQ ID NO:1, 3, 4, 6, 18, 20, 21, 23, 24, or 26, thereby leading to changes in the amino acid sequence of the encoded TLT-1 proteins, without altering the functional ability of the TLT-1 proteins. For example, nucleotide substitutions leading to amino acid substitutions at “non-essential” amino acid residues can be made in the sequence of SEQ ID NO:1, 3, 4, 6, 18, 20, 21, 23, 24, or 26. A “non-essential” amino acid residue is a residue that can be altered from the wild-type sequence of TLT-1 (e.g., the sequence of SEQ ID NO:2, 5, 19, 22, or 25) without altering the biological activity, whereas an “essential” amino acid residue is required for biological activity. For example, amino acid residues that are conserved among the TLT-1 proteins of the present invention, e.g., those present in an ITIM, are predicted to be particularly unamenable to alteration. Furthermore, additional amino acid residues that are conserved between the TLT-1 proteins of the present invention and/or other members of the TLT-1 family are not likely to be amenable to alteration.

Accordingly, another aspect of the invention pertains to nucleic acid molecules encoding TLT-1 proteins that contain changes in amino acid residues that are not essential for activity. Such TLT-1 proteins differ in amino acid sequence from SEQ ID NO:25, 19, 22, or 25, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 81%, 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to SEQ ID NO:25, 19, 22, or 25.

An isolated nucleic acid molecule encoding a TLT-1 protein identical to the protein of SEQ ID NO:25, 19, 22, or 25 can be created by introducing one or more nucleotide substitutions, additions or deletions into the nucleotide sequence of SEQ ID NO:1, 3, 4, 6, 18, 20, 21, 23, 24, or 26 such that one or more amino acid substitutions, additions or deletions are introduced into the encoded protein. Mutations can be introduced into SEQ ID NO:1, 3, 4, 6, 18, 20, 21, 23, 24, or 26 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a TLT-1 protein is preferably replaced with another amino acid residue from the same side chain family. Alternatively, in another embodiment, mutations can be introduced randomly along all or part of a TLT-1 coding sequence, such as by saturation mutagenesis, and the resultant mutants can be screened for TLT-1 biological activity to identify mutants that retain activity. Following mutagenesis of SEQ ID NO:1, 3, 4, 6, 18, 20, 21, 23, 24, or 26, the encoded protein can be expressed recombinantly and the activity of the protein can be determined.

In a preferred embodiment, a mutant TLT-1 protein can be assayed for the ability to 1) interact with a TLT-1 target molecule (e.g., a second TLT-1 molecule or a non-TLT-1 molecule such as a TLT-1 specific antibody, a TLT-1 ligand, a cell-surface protein, a Src family member, SHP-1, SHP-2, SHP1, an SH2 domain containing protein an SH3 domain containing protein, and/or a WW domain containing protein); 2) modulate megakaryocyte differentiation; 3) modulate platelet differentiation and/or production (thrombopoiesis)-4) modulate platelet activity-5) modulate intra- or inter-cellular signaling; 6) localize to platelet and or megakaryocyte alpha granules: 7) modulate platelet and/or megakaryocyte granule formation and/or sorting; 8) localize to the platelet and/or megakaryocte cell surface, 9) modulate platelet interaction with and/or adhesion to the extracellular matrix and/or basement membrane;, 10) modulate blood clotting; 11) modulate bleeding; 12) modulate immune responses; 13) modulate activation of neutrophils and/or other leukocytes; 14) modulate dendritic cell maturation and/or function; and/or 15) modulate cellular proliferation.

In addition to the nucleic acid molecules encoding TLT-1 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TLT-1 coding strand, or to only a portion thereof. In one embodiment, an antisense nucleic acid molecule is antisense to a “coding region” of the coding strand of a nucleotide sequence encoding a TLT-1. The term “coding region” refers to the region of the nucleotide sequence comprising codons which are translated into amino acid residues (e.g., the coding region of mouse TLT-1 corresponds to SEQ ID NO:3, the coding region of the RAW 264.7 splice variant corresponds to SEQ ID NO:20, the coding region of the second mouse splice variant corresponds to SEQ ID NO:23, the coding region of human TLT-1 corresponds to SEQ ID NO:6, and the coding region of the human splice variant corresponds to SEQ ID NO:26). In another embodiment, the antisense nucleic acid molecule is antisense to a “noncoding region” of the coding strand of a nucleotide sequence encoding TLT-1. The term “noncoding region” refers to 5′ and 3′ sequences which flank the coding region that are not translated into amino acids (i.e., also referred to as 5′ and 3′ untranslated regions).

Given the coding strand sequences encoding TLT-1 disclosed herein (e.g., SEQ ID NOs:3, 6, 20, 23, and 26), antisense nucleic acids of the invention can be designed according to the rules of Watson and Crick base pairing. The antisense nucleic acid molecule can be complementary to the entire coding region of TLT-1 mRNA, but more preferably is an oligonucleotide which is antisense to only a portion of the coding or noncoding region of TLT-1 mRNA. For example, the antisense oligonucleotide can be complementary to the region surrounding the translation start site of TLT-1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a TLT-1 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave TLT-1 mRNA transcripts to thereby inhibit translation of TLT-1 mRNA. A ribozyme having specificity for a TLT-1-encoding nucleic acid can be designed based upon the nucleotide sequence of a TLT-1 cDNA disclosed herein (i.e., SEQ ID NO:1, 3, 4, 6, 18, 20, 21, 23, 24, or 26). For example, a derivative of a Tetrahymena L-19 IVS RNA can be constructed in which the nucleotide sequence of the active site is complementary to the nucleotide sequence to be cleaved in a TLT-1-encoding mRNA. See, e.g., Cech et al. U.S. Pat. No. 4,987,071; and Cech et al. U.S. Pat. No. 5,116,742. Alternatively, TLT-1 mRNA can be used to select a catalytic RNA having a specific ribonuclease activity from a pool of RNA molecules. See, e.g., Bartel, D. and Szostak, J. W. (1993) Science 261:1411-1418.

Alternatively, TLT-1 gene expression can be inhibited by targeting nucleotide sequences complementary to the regulatory region of TLT-1 (e.g., the TLT-1 promoter and/or enhancers, or the untranslated regions e.g., nucleotides 1-21 of SEQ ID NO:1 or nucleotides 988-1220 of SEQ ID NO:1) to form triple helical structures that prevent transcription of the TLT-1 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In yet another embodiment, the TLT-1 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA. 93:14670-675.

PNAs of TLT-1 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TLT-1 nucleic acid molecules can also be used in the analysis of single base pair mutations in a gene, (e.g., by PNA-directed PCR clamping); as ‘artificial restriction enzymes’ when used in combination with other enzymes (e.g., Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of TLT-1 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of TLT-1 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17:5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Biotechniques 6:958-976) or intercalating agents (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous TLT-1 gene within a cell line or microorganism may be modified by inserting a heterologous DNA regulatory element into the genome of a stable cell line or cloned microorganism such that the inserted regulatory element is operatively linked with the endogenous TLT-1 gene. For example, an endogenous TLT-1 gene which is normally “transcriptionally silent”, i.e., a TLT-1 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous TLT-1 gene may be activated by insertion of a promiscuous regulatory element that works across cell types.

A heterologous regulatory element may be inserted into a stable cell line or cloned microorganism, such that it is operatively linked with an endogenous TLT-1 gene, using techniques, such as targeted homologous recombination, which are well known to those of skill in the art, and described, e.g., in Chappel, U.S. Pat. No. 5,272,071; PCT publication No. WO 91/06667, published May 16, 1991.

Expression of TLT-1 can also be modulated using small interfering RNA (siRNA) in RNA interference (RNAi). RNAi as a protecting mechanism against invasion by foreign genes was first described in C. elegans and has subsequently been demonstrated in diverse eukaryotes such as insects, plants, fungi and vertebrates. RNAi is the mechanism of sequence-specific, post-transcriptional gene silencing initiated by double-stranded RNAs (dsRNA) homologous to the gene being suppressed. dsRNAs are processed by Dicer, a cellular ribonuclease III, to generate duplexes of about 21 nt with 3′-overhangs (small interfering RNA, siRNA) which mediate sequence-specific mRNA degradation. In mammalian cells siRNA molecules are capable of specifically silencing gene expression without induction of the unspecific interferon response pathway. Thus, siRNAs are a powerful alternative to other genetic tools such as antisense oligonucleotides and ribozymes to analyze loss-of-function phenotypes. Application of siRNA duplexes to interfere with the expression of a specific gene requires knowledge of target accessibility, highly effective delivery of siRNAs into target cells and for some applications long-term siRNA expression. Effective strategies to deliver siRNAs to target cells in cell culture include transduction by physical or chemical transfection. An alternative strategy uses the endogenous expression of siRNAs by various Pol III promoter expression cassettes that allow transcription of functional siRNAs or their precursors. Kits for producing siRNAs are widely available from commercial companies. Decriptions of RNAi and siRNAs can be found, for example, in Scherr, M. et al. (2003) Curr Med Chem. 10(3):245-56; Shuey, D. J. et al. (2002) Drug Discov. Today 7(20):1040-6; Shi Y. (2003) Trends Genet. 19(1):9-12; Morita, T. and Yoshida, K. (2002) Tanpakushitsu Kakusan Koso 47(14):1839-45; Famulok, M. and Verma, S. (2002) Trends Biotechnol. 20(11):462-6; Timmons L. (2002) Mol Cell. 10(3):435-7; Kitabwalla, M, and Ruprecht, R. M.(2002) N. Engl. J. Med. 347(17):1364-7; McManus, M. T. and Sharp, P. A. (2002) Nat. Rev. Genet. 3(10):737-47; Micura, R. (2002) Angew Chem. Int. Ed. Engl. 41(13):2265-9; Lin, S. L. and Ying, S. Y. (2001) Curr. Cancer Drug Targets 1(3):241-7; Voinnet O. (2002) Curr. Opin. Plant Biol. 5(5):444-51; Cullen, B. R. (2002) Nat. Immunol. 3(7):597-9; Hudson, D. F. et al. (2002) Trends Cell Biol. 2(6):281-7; Mlotshwa, S. et al. Plant Cell 14 Suppl:S289-301; Ahlquist, P. (2002) Science 296(5571):1270-3; Ullu, E. et al. (2002) Philos. Trans. R. Soc. Lond. B. Biol. Sci. 357(1417):65-70; Inoue, H. (2001) Seikagaku 73(12):1444-7; Tuschl, T. (2001) Chembiochem. 2(4):239-45; and U.S. Patent Application Publication Nos. 20020086356, 20020132788, 20020173478.

II. Isolated TLT-1 Proteins and Anti-TLT-1 Antibodies

One aspect of the invention pertains to isolated TLT-1 proteins, and biologically active portions thereof, as well as polypeptide fragments suitable for use as immunogens to raise anti-TLT-1 antibodies. In one embodiment, native TLT-1 proteins can be isolated from cells or tissue sources by an appropriate purification scheme using standard protein purification techniques. In another embodiment, TLT-1 proteins are produced by recombinant DNA techniques. Alternative to recombinant expression, a TLT-1 protein or polypeptide can be synthesized chemically using standard peptide synthesis techniques.

An “isolated” or “purified” protein or biologically active portion thereof is substantially free of cellular material or other contaminating proteins from the cell or tissue source from which the TLT-1 protein is derived, or substantially free from chemical precursors or other chemicals when chemically synthesized. The language “substantially free of cellular material” includes preparations of TLT-1 protein in which the protein is separated from cellular components of the cells from which it is isolated or recombinantly produced. In one embodiment, the language “substantially free of cellular material” includes preparations of TLT-1 protein having less than about 30% (by dry weight) of non-TLT-1 protein (also referred to herein as a “contaminating protein”), more preferably less than about 20% of non-TLT-1 protein, still more preferably less than about 10% of non-TLT-1 protein, and most preferably less than about 5% non-TLT-1 protein. When the TLT-1-protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of TLT-1 protein in which the protein is separated from chemical precursors or other chemicals which are involved in the synthesis of the protein. In one embodiment, the language “substantially free of chemical precursors or other chemicals” includes preparations of TLT-1 protein having less than about 30% (by dry weight) of chemical precursors or non-TLT-1 chemicals, more preferably less than about 20% chemical precursors or non-TLT-1 chemicals, still more preferably less than about 10% chemical precursors or non-TLT-1 chemicals, and most preferably less than about 5% chemical precursors or non-TLT-1 chemicals.

As used herein, a “biologically active portion” of a TLT-1 protein includes a fragment of a TLT-1 protein which participates in an interaction between a TLT-1 molecule and a non-TLT-1 molecule. Biologically active portions of a TLT-1 protein include peptides comprising amino acid sequences sufficiently identical to or derived from the amino acid sequence of the TLT-1 protein, e.g., the amino acid sequence shown in SEQ ID NO:2, 5, 19, 22, or 25, which include less amino acids than the full length TLT-1 proteins, and exhibit at least one activity of a TLT-1 protein. Typically, biologically active portions comprise a domain or motif with at least one activity of the TLT-1 protein, e.g., 1) interaction with a TLT-1 target molecule (e.g., a second TLT-1 molecule, or a non-TLT-1 inolecule such as a TLT-1 specific antibody, a TLT-1 ligand, a cell-surface protein, a Src family member, SHP-1, SHP-2, SHIP-1, an SH2 domain containing protein, an SH3 domain containing protein, and/or a WW domain containing protein); 2) modulation of megakaryocyte differentiation; 3) modulation of platelet differentiation and/or production (thrombopoiesis); 4) modulation of platelet activity; 5) modulation of intra- or inter-cellular signaling; 6) localization to platelet and/or megakaryocyte alpha granules; 7) modulation of platelet and/or megakaryocyte granule formation and/or sorting; 8) localization to the platelet and/or niegakaryocyte cell surface; 9) modulation of platelet interaction with and/or adhesion to the extracellular matrix and/or basement membrane; 10) modulation of blood clotting; 11) modulation of bleeding; 12) modulation of immune responses; 13) modulation of activation of neutrophils and/or other leukocytes; 14) modulation of dendritic cell maturation and/or function; and/or 15) modulation of cellular proliferation. A biologically active portion of a TLT-1 protein can be a polypeptide which is, for example, 5, 6, 7, 8, 9, 10, 15, 20, 25, 30, 35, 40, 45, 50, 75, 100, 125, 150, 175, 200, 225, 250, 275, 300, 305, 310, 311, 312, 313, 314, 315, 320, 321 or more amino acids in length. Biologically active portions of a TLT-1 protein can be used as targets for developing agents which modulate a TLT-1 mediated activity, e.g., a platelet associated activity.

In one embodiment, a biologically active portion of a TLT-1 protein comprises at least one transmembrane domain. It is to be understood that a preferred biologically active portion of a TLT-1 protein of the present invention may contain at least one transmembrane domain a signal peptide domain, and IgV domain, a cysteine residue, an extracellular domain, a cytoplasmic domain, a polyproline-rich region, tin ITIM, and an O-glycosylation site. Moreover, other biologically active portions, in which other regions of the protein are deleted, can be prepared by recombinant techniques and evaluated for one or more of the functional activities of a native TLT-1 protein.

In one embodiment, a TLT-1 protein of the invention is a dominant negative TLT-1 protein. As used herein, a “dominant negative” includes a protein or polypeptide that, when expressed in the cell, interferes with, downregulates, and/or inactivates the conesponding wild-type protein expressed in the cell. For example, a dominant negative TLT-1 protein may include a extracellular domain (e.g., the ligand-binding domain) and a transmembrane domain, but no cytoplasmic domain. In another embodiment, a dominant negative TLT-1 protein may include a cytoplasmic domain and a transmembrane domain, but no extracellular domain. Such dominant negative proteins, may act, for example, by binding TLT-1 target molecules without being able to transmit an intracellular signal, leaving no target molecules available for binding to the wild-type protein.

In a preferred embodiment, the TLT-1 protein has an amino acid sequence shown in SEQ ID NO:25, 19, 22, or 25. In other embodiments, the TLT-1 protein is substantially identical to SEQ ID NO:25, 19, 22, or 25, and retains the functional activity of the protein of SEQ ID NO:25, 19, 22, or 25, yet differs in amino acid sequence due to natural allelic variation or mutagenesis, as described in detail in subsection I above. Accordingly, in another embodiment, the TLT-1 protein is a protein which comprises an amino acid sequence at least about 50%, 55%, 60%, 65%, 70%, 75%, 80%, 81%, 85%, 90%, 95%, 96%, 97%, 98%, 99%, 99.1%, 99.2%, 99.3%, 99.4%, 99.5%, 99.6%, 99.7%, 99.8%, 99.9%, 99.99% or more identical to SEQ ID NO:25, 19, 22, or 25.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the mouse TLT-1 amino-acid sequence of SEQ ID NO:2 having 322 amino acid residues, at least 97, preferably at least 129, more preferably at least 161, even more preferably at least 193, and even more preferably at least 225, 258, 290 or more amino acid residues are aligned; when aligning a second sequence to the human TLT-1 amino acid sequence of SEQ ID NO:5 having 311 amino acid residues, at least 93, preferably at least 124, more preferably at least 156, even more preferably at least 187, and even more preferably at least 218, 249, 280 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available online through the website of the Genetics Computer Group), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online through the website of the Genetics Computer Group), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TLT-1 nucleic acid molecules of the invention. BLAST protein searches can be performed with the XBLAST program, score=100, wordlength=3 to obtain amino acid sequences homologous to TLT-1 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See the website of the National Center for Biotechnology Information.

The invention also provides TLT-1 chimeric or fusion proteins. As used herein, a TLT-1 “chimeric protein” or “fusion protein” comprises a TLT-1 polypeptide operatively linked to a non-TLT-1 polypeptide. A “TLT-1 polypeptide” refers to a polypeptide having an amino acid sequence corresponding to a TLT-1 molecule, whereas a “non-TLT-1 polypeptide” refers to a polypeptide having an amino acid sequence corresponding to a protein which is not substantially homologous to the TLT-1 protein, e.g., a protein which is different from the TLT-1 protein and which is derived from the same or a different organism. Within a TLT-1 fusion protein the TLT-1 polypeptide can correspond to all or a portion of a TLT-1 protein. In a preferred embodiment, a TLT-1 fusion protein comprises at least one biologically active portion of a TLT-1 protein. In another preferred embodiment, a TLT-1 fusion protein comprises at least two biologically active portions of a TLT-1 protein. For example, in a preferred embodiment, the TLT-1 portion of a TLT-1 fusion protein comprises only an extracellular domain or only an intracellular domain. In another embodiment, the TLT-1 portion of a TLT-1 fusion protein comprises a extracellular domain and a transmembrane domain without an intracellular domain, or an intracellular domain and a transmembrane domain without an extracellular domain. Within the fusion protein, the term “operatively linked” is intended to indicate that the TLT-1 polypeptide and the non-TLT-1 polypeptide are fused in-frame to each other. The non-TLT-1 polypeptide can be fused to the N-terminus or C-terminus of the TLT-1 polypeptide.

For example, in one embodiment, the fusion protein is a GST-TLT-1 fusion protein in which the TLT-1 sequences are fused to the C-terminus of the GST sequences. In other embodiments, TLT-1 fusion proteins contain polyhistidine tags (e.g., 6 histidine residues), myc tags, or other peptides known in the art as “epitope tags”. Such fusion proteins can facilitate the purification and/or detection of recombinant TLT-1.

In another embodiment, the fusion protein is a TLT-1 protein containing a heterologous signal sequence at its N-terminus. In certain host cells (e.g., mammalian host cells), expression and/or secretion of TLT-1 can be increased through use of a heterologous signal sequence.

In a preferred embodiment, the fusion protein is an Ig-TLT-1 fusion protein in which the TLT-1 sequences are fused to a portion of an Ig molecule. The Ig portion of the fusion protein can include and immunoglobulin constant region, e.g., a human Cγ1 domain or a Cγ4 domain (e.g., the hinge, CH2, and CH3 regions of human IgCγ1 or human IgCγ4 (see, e.g., Capon et al., U.S. Pat. Nos. 5,116,964; 5,580,756; 5,844,095, and the like, incorporated herein by reference). A resulting fusion protein may have altered TLT-1 solubility, binding affinity, stability and/or valency (i.e., the number of binding sites per molecule) and may increase the efficiency of protein purification.

Particularly preferred TLT-1 Ig fusion proteins include an extracellular domain portion of TLT-1 coupled to an immunoglobulin constant region (e.g., the Fc region). The immunoglobulin constant region may contain genetic modifications which reduce or eliminate effector activity inherent in the immunoglobulin structure. For example, DNA encoding an extracellular portion of a TLT-1 polypeptide can be joined to DNA encoding the hinge, CH2, and CH3 regions of human IgGγ1 and/or IgGγ4 modified by site-directed mutagenesis, e.g., as taught in WO 97/28267.

The TLT-1 fusion proteins of the invention can be incorporated into pharmaceutical compositions and administered to a subject in vivo. The TLT-1 fusion proteins can be used to affect the bioavailability of a TLT-1 ligand or binding partner. Use of TLT-1 fusion proteins may be useful therapeutically for the treatment of disorders caused by, for example, (i) aberrant modification or mutation of a gene encoding a TLT-1 protein; (ii) mis-regulation of the TLT-1 gene; and (iii) aberrant post-translational modification of a TLT-1 protein.

Moreover, the TLT-1-fusion proteins of the invention can be used as immunogens to produce anti-TLT-1 antibodies in a subject, to purify TLT-1 ligands and in screening assays to identify molecules which inhibit the interaction of TLT-1 with a TLT-1 ligand or binding partner.

Preferably, a TLT-1 chimeric or-fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A TLT-1-encoding nucleic acid can be cloned into such an expression vector such that the fusion moiety is linked in-frame to the TLT-1 protein.

The present invention also pertains to variants of the TLT-1 proteins which function as either TLT-1 agonists (mimetics) or as TLT-1 antagonists. Variants of the TLT-1 proteins can be generated by mutagenesis, e.g., discrete point mutation or truncation of a TLT-1 protein. An agonist of the TLT-1 proteins can retain substantially the same, or a subset, of the biological activities of the naturally occurring form of a TLT-1 protein. An antagonist of a TLT-1 protein can inhibit one or more of the activities of the naturally occurring form of the TLT-1 protein by, for example, competitively modulating a TLT-1-mediated activity of a TLT-1 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TLT-1 protein.

In one embodiment, variants of a TLT-1 protein which function as either TLT-1 agonists (mimetics) or as TLT-1 antagonists can be identified by screening combinatorial libraries of mutants, e.g., truncation mutants, of a TLT-1 protein for TLT-1 protein agonist or antagonist activity. In one embodiment, a variegated library of TLT-1 variants is generated by combinatorial mutagenesis at the nucleic acid level and is encoded by a variegated gene library. A variegated library of TLT-1 variants can be produced by, for example, enzymatically ligating a mixture of synthetic oligonucleotides into gene sequences such that a degenerate set of potential TLT-1 sequences is expressible as individual polypeptides, or alternatively, as a set of larger fusion proteins (e.g., for phage display) containing the set of TLT-1 sequences therein. There are a variety of methods which can be used to produce libraries of potential TLT-1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TLT-1 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477.

In addition, libraries of fragments of a TLT-1 protein coding sequence can be used to generate a variegated-population of TLT-1 fragments for screening and subsequent selection of variants of a TLT-1 protein. In one embodiment, a library of coding sequence fragments can be generated by treating a double stranded PCR fragment of a TLT-1 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TLT-1 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TLT-1 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TLT-1 variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated TLT-1 library. For example, a library of expression vectors can be transfected into a cell line, e.g., a myeloid cell line, which ordinarily responds to a TLT-1 ligand in a particular TLT-1 ligand-dependent manner. The transfected cells are then contacted with a TLT-1 ligand and the effect of expression of the mutant on, e.g., signaling by TLT-1 can be detected. Plasmid DNA can then be recovered from the cells which score for inhibition, or alternatively, potentiation of signaling by the TLT-1 ligand, and the individual clones further characterized.

An isolated TLT-1 protein, or a portion or fragment thereof, can be used as an immunogen to generate antibodies that bind TLT-1 using standard techniques for polyclonal and monoclonal antibody preparation. A full-length TLT-1 protein can be used or, alternatively, the invention provides antigenic peptide fragments of TLT-1 for use as immunogens. The antigenic peptide of TLT-1 comprises at least 8 amino acid residues of the amino acid sequence shown in SEQ ID NO:25, 19, 22, or 25 and encompasses an epitope of TLT-1 such that an antibody raised against the peptide forms a specific immune complex with the TLT-1 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of TLT-1 that are located on the surface of the protein, e.g., hydrophilic regions, as well as regions with high antigenicity.

A TLT-1 immunogen typically is used to prepare antibodies by immunizing a suitable subject, (e.g., rabbit, goat, mouse or other mammal) with the immunogen. An appropriate immunogenic preparation can contain, for example, recombinantly expressed TLT-1 protein or a chemically synthesized TLT-1 polypeptide. The preparation can further include an adjuvant, such as Freund's complete or incomplete adjuvant, or similar immunostimulatory agent. Immunization of a suitable subject with an immunogenic TLT-1 preparation induces a polyclonal anti-TLT-1 antibody response.

Accordingly, another aspect of the invention pertains to anti-TLT-1 antibodies. The term “antibody” as used herein refers to immunoglobulin molecules and immunologically active portions of immunoglobulin molecules, i.e., molecules that contain an antigen binding site which specifically binds (immunoreacts with) an antigen, such as a TLT-1. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind TLT-1 molecules. The term “monoclonal antibody” or “monoclonal antibody composition”, as used herein, refers to a population of antibody molecules that contain only one species of an antigen binding site capable of immunoreacting with a particular epitope of TLT-1. A monoclonal antibody composition thus typically displays a single binding affinity for a particular TLT-1 protein with which it immunoreacts.

Polyclonal anti-TLT-1 antibodies can be prepared as described above by immunizing a suitable subject with a TLT-1 immunogen. The anti-TLT-1 antibody titer in the immunized subject can be monitored over time by standard techniques, such as with an enzyme linked immunosorbent assay (ELISA) using immobilized TLT-1. If desired, the antibody molecules directed against TLT-1 can be isolated from the mammal (e.g., from the blood) and further purified by well known techniques, such as protein A chromatography to obtain the IgG fraction. At an appropriate time after immunization, e.g., when the anti-TLT-1 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al.,(1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y (1980); E. A. Lerner (1981) Yale J. Biol. Med. 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a TLT-1 immunogen as described above, and the culture supernatants of the resulting hybridoma cells are screened to identify a hybridoma producing a monoclonal antibody that binds TLT-1.

Any of the many well known protocols used for fusing lymphocytes and immortalized cell lines can be applied for the purpose of generating an anti-TLT-1 monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TLT-1, e.g., using a standard ELISA assay.

Alternative to preparing monoclonal antibody-secreting hybridomas, a monoclonal anti-TLT-1 antibody can be identified and isolated by screening a recombinant combinatorial immunoglobulin library (e.g., an antibody phage display library) with TLT-1 to thereby isolate immunoglobulin library members that bind TLT-1. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-TLT-1 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-TLT-1 antibody (e.g., monoclonal antibody) can be used to isolate TLT-1 by standard techniques, such as affinity chromatography or immunoprecipitation. An anti-TLT-1 antibody can facilitate the purification of natural TLT-1 from cells and of recombinantly produced TLT-1 expressed in host cells. Moreover, an anti-TLT-1 antibody can be used to detect TLT-1 protein (e.g., in a cellular lysate or cell supernatant) in order to evaluate the abundance and pattern of expression of the TLT-1 protein. Anti-TLT-1 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

II. Recombinant Expression Vectors and Host Cells

Another aspect of the invention pertains to vectors, preferably expression vectors, containing a nucleic acid encoding a TLT-1 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., TLT-1 proteins, mutant forms of TLT-1 proteins, fusion proteins, and the like).

The recombinant expression vectors of the invention can be designed for expression of TLT-1 proteins in prokaryotic or eukaryotic cells. For example, TLT-1 proteins can be expressed in bacterial cells such as E. coli, insect cells (using baculovirus expression vectors) yeast cells or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression.-vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in TLT-1 activity assays, (e.g., direct assays or competitive assays described in detail below), or to generate antibodies specific for TLT-1 proteins, for example. In a preferred embodiment, a TLT-1 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al. (1990) Methods Enzymol. 185:60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S. (1990) Methods Enzymol. 185:119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the TLT-1 expression vector is a yeast expression vector. Examples of vectors for expression in yeast S. cerevisiae include pYepSec1 (Baldari, et al., (1987) EMBO J. 6:229-234), pMFa (Kuran and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, TLT-1 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the platelet-specific promoters from the genes CD41 and CD62, the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TLT-1 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a TLT-1 nucleic acid molecule of the invention is introduced, e.g., a TLT-1 nucleic acid molecule within a recombinant expression vector or a TLT-1 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TLT-1 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a TLT-1 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a TLT-1 protein. Accordingly, the invention further provides methods for producing a TLT-1 protein using the host cells of the invention. In one embodiment, the method comprises culturing the host cell of the invention (into which a recombinant expression vector encoding a TLT-1 protein has been introduced) in a suitable medium such that a TLT-1 protein is produced. In another embodiment, the method further comprises isolating a TLT-1 protein from the medium or the host cell.

The host cells of the invention can also be used to produce non-human transgenic animals. For example, in one embodiment, a host cell of the invention is a fertilized oocyte or an embryonic stem cell into which TLT-1-coding sequences have been introduced. Such host cells can then be used to create non-human transgenic animals in which exogenous TLT-1 sequences have been introduced into their genome or homologous recombinant animals in which endogenous TLT-1 sequences have been altered. Such animals are useful for studying the function and/or activity of a TLT-1 and for identifying and/or evaluating modulators of TLT-1 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous TLT-1 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a TLT-1-encoding nucleic acid into the male pronuclei of a fertilized oocyte, e.g., by microinjection, retroviral infection, and allowing the oocyte to develop in a pseudopregnant female foster animal. The TLT-1 cDNA sequence of SEQ ID NO:1, 3, 4, 6, 18, 20, 21, 23, 24, or 26 can be introduced as a transgene into the genome of a non-human animal. Alternatively, a TLT-1 gene homologue, such as another TLT-1 family member, can be isolated based on hybridization to the TLT-1 cDNA sequences of SEQ ID NO:1, 3, 4, 6, 18, 20, 21, 23, 24, or 26 (described further in subsection I above) and used as a transgene. Intronic sequences and polyadenylation signals can also be included in the transgene to increase the efficiency of expression of the transgene. A tissue-specific regulatory sequence(s) can be operably linked to a TLT-1 transgene to direct expression of a TLT-1 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a TLT-1 transgene in its genome and/or expression of TLT-1 mRNA in tissues or cells of the animals. A transgenic founder animal can then be used to breed additional animals carrying the transgene. Moreover, transgenic animals carrying a transgene encoding a TLT-1 protein can further be bred to other transgenic animals carrying other transgenes.

To create a homologous recombinant animal, a vector is prepared which contains at least a portion of a TLT-1 gene into which a deletion, addition or substitution has been introduced to thereby alter, e.g., functionally disrupt, the TLT-1 gene. The TLT-1 gene can be a human gene (e.g., the cDNA of SEQ ID NO:4, 6, 24, or 26), but more preferably, is a non-human homologue of a human TLT-1 gene. For example, the mouse TLT-1 sequence of SEQ ID NO:1, 3, 18, 20, 21, or 23 can be used to construct a homologous recombination nucleic acid molecule, e.g., a vector, suitable for altering an endogenous TLT-1 gene in the mouse genome. In a preferred embodiment, the homologous recombination nucleic acid molecule is designed such that, upon homologous recombination, the endogenous TLT-1 gene is functionally disrupted (i.e., no longer encodes a functional protein; also referred to as a “knock out” vector). Alternatively, the homologous recombination nucleic acid molecule can be designed such that, upon homologous recombination, the endogenous TLT-1 gene is mutated or otherwise altered but still encodes functional protein (e.g., the upstream regulatory region can be altered to thereby alter the expression of the endogenous TLT-1 protein). In the homologous recombination nucleic acid molecule, the altered portion of the TLT-1 gene is flanked at its 5′ and 3′ ends by additional nucleic acid sequence of the TLT-1 gene to allow for homologous recombination to occur between the exogenous TLT-1 gene carried by the homologous recombination nucleic acid molecule and an endogenous TLT-1 gene in a cell, e.g., an embryonic stem cell. The additional flanking TLT-1 nucleic acid sequence is of sufficient length for successful homologous recombination with the-endogenous gene. Typically, several kilobases of flanking DNA (both at the 5′ and 3′ ends) are included in the homologous recomibination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced TLT-1 gene has homologously recombined with the endogenous TLT-1 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ -cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Curr. Opin. Biotechnol. 2:823-829 and in PCT International Publication Nos. WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The TLT-1 nucleic acid molecules, fragments of TLT-1 proteins, and anti-TLT-1 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a TLT-1 protein or an anti-TLT-1 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In certain embodiments of the invention, a modulator of TLT-1 activity is administered in combination with other agents (e.g., a small molecule), or in conjunction with another, complementary treatment regime. For example, in one embodiment, a modulator of TLT-1 activity is used to treat a platelet associated disorder. Accordingly, modulation of TLT-1 activity may be used in conjunction with another agent used to treat the disorder. For example, a TLT-i modulator may be used in conjunction with other agents used to treat clotting or bleeding disorders, for example, thrombopoiesis-inducing agents (e.g., IL-11, thrombopoietin (TPO), anti-platelet agents (e.g., aspirin, non-steroidal anti-inflammatory drugs (e.g., ibuprofen, naproxen sodium), dipyridamole (Persantine), Aggrenox, ticlopidine (Ticlid), clopidogrel (Plavix), and GPIIB/IIIA inhibitors), anticoagulants (e.g., warfarin (Coumadin), heparin, low-molecular-weight heparin, danaparoid, hirudin, lepirudin (Refludan), bivalirudin (Hirulog), and Argatroban), and thrombolytic medications (also referred to as “clot-busters”, e.g., streptokinase, urokinase-type plasminogen activator (UPA), and tissue-type plasminogen activator (TPA)).

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiaamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (IL-1), interleukin-2 (IL-2), interleukin-6 (IL-6), interleukin-11 (IL-11), granulocyte macrophage colony stimulating factor (GM-CSF), granulocyte colony stimulating factor (G-CSF), thrombopoietin (TPO), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The pharmaceutical compositions can be included in a container, pack, or dispenser together with instructions for administration.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In a preferred embodiment, cells (e.g., bone marrow cells or blood cells such as platelets, leukocytes, and/or other blood cells) are removed from a subject (e.g., a human, a mouse, or other mammal), and a gene therapy vector containing a nucleic acid molecule of the invention is delivered to the cells ex vivo. After deliver of the gene therapy vector to the cells, the cells are returned to the subject.

Viral vectors include, for example, recombinant retroviruses, adenovirus, adeno-associated virus, and herpes simplex virus-1. Retrovirus vectors and adeno-associated virus vectors are generally understood to be the recombinant gene delivery system of choice for the transfer of exogenous genes in vivo, articularly into humans.

A major prerequisite for the use of viruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, a recombinant retrovirus can be constructed in which part of the retroviral coding sequence (gag, pol, env) is replaced by a gene of interest rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include φCrip, φCre, φ2 and φAm.

Furthermore, it has been shown that it is possible to limit the infection spectrum of retroviruses and consequently of retroviral-based vectors, by modifying the viral packaging proteins on the surface of the viral particle (see, for example PCT publications WO 93/25234 and WO 94/06920). For instance, strategies for the modification of the infection spectrum of retroviral vectors include: coupling antibodies specific for cell surface antigens to the viral env protein (Roux et al. (1989) Proc. Natl Acad. Sci. USA 86:9079-9083; Julan et al. (1992) J. Gen. Virol. 73:3251-3255; and Goud et al. (1983) Virology 163:251-254); or coupling cell surface receptor ligands to the viral env proteins (Neda et al. (1991) J. Biol. Chem. 266:14143-14146). Coupling can be in the form of the chemical cross-linking with a protein or other variety (e g. lactose to convert the env protein to an asialoglycoproicin), as well as by generating fusion proteins (e.g. single-chain antibody/env fusion proteins). Thus, in a specific embodiment of the invention, viral particles containing a nucleic acid molecule containing a gene of interest operably linked to appropriate regulatory elements, are modified for example according to the methods described above, such that they can specifically target subsets of liver cells. For example, the viral particle can be coated with antibodies to surface molecule that are specific to certain types of liver cells. This method is particularly useful when only specific subsets of liver cells are desired to be transfected.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berlcner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the gene of interest comprised in the nucleic acid molecule can be under control of, for example, the E1A promoter, the major late promoter (MLP) and associated leader sequences, the E3 promoter, or exogenously added promoter sequences.

Yet another viral vector system useful for delivery of a nucleic acid molecule comprising a gene of interest is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics Microbiol. Immnunol. 158:97-129). Adeno-associated viruses exhibit a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as few as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into T cells. A variety of nucleic acids have been introduced into different cell types using AAV vectors (see for example Hermonat et al. (1984) Proc. Natl. Acad. Sci. USA 81:6466-6470; Tratschin et al. (1985) Mol Cell. Biol. 4:2072-208 1; Wondisford et al. (1988) Mol. Endocrinol. 2:32-39; Tratschin et al. (1984) J. Virol. 51:611-619; and Flotte et al. (1993) J. Biol. Chem. 268:3781-3790). Other viral vector systems that may have application in gene therapy have been derived from herpes virus, vaccinia virus, and several RNA viruses.

Other methods relating to the use of viral vectors in gene therapy can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11: 179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N. Y Acad Sci. 716:90-101; Strayer, D. S. (1999) Expert Opin. Invetig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a TLT-1 protein of the invention has one or more of the following activities: 1) it interacts with a TLT-1 target molecule (e.g., a second TLT-1 molecule, or a non-TLT-1 molecule such as a TLT-1 specific autibody, a TLT-1 ligand, a cell-surface protein, a Src family nmember, SHP-1, SHP-2, SHIP-1, an SH2 domain containing protein, an SH3 domain containing protein, and/or a WW domain containing protein); 2) it modulates megakaryocyte differentiation; 3) it modulates platelet differentiation and/or production (thrombopoiesis); 4) it modulates platelet activity; 5) it modulates intra- or inter-cellular signaling; 6) it localizes to platelet and/or megakaryocyte alpha granules; 7) it modulates platelet and/or megkaryocyte granule formation and/or sorting; 8) it localizes to the platelet and/or megakaryocyte cell surface; 9) it modulates platelet interaction with and/or adhesion to the extracellular matrix and/or basement membrane; 10) it modulates blood clotting; 11) it modulates bleeding; 12) it modulates immune responses; 13) it modulates activation of neutrophils and/or other leukocytes; 14) it modulates dendritic cell maturation and/or function; and/or 15) it modulates cellular proliferation.

The isolated nucleic acid molecules of the invention can be used, for example, to express TLT-1 protein (e.g., via a recombinant expression vector in a host cell in gene therapy applications), to detect TLT-1 mRNA (e.g., in a biological sample) or a genetic alteration in a TLT-1 gene, and to modulate TLT-1 activity, as described further below. The TLT-1 proteins can be used to treat disorders characterized by insufficient or excessive production of a TLT-1 target molecule or production of TLT-1 inhibitors. In addition, the TLT-1 proteins can be used to screen for naturally occurring TLT-1 ligands and binding partners, to screen for drugs or compounds which modulate TLT-1 activity, as well as to treat disorders characterized by insufficient or excessive production of TLT-1 protein or production of TLT-1 protein forms which have decreased, aberrant or unwanted activity compared to TLT-1 wild type protein (e.g., platelet-associated disorders). Moreover, the anti-TLT-1 antibodies of the invention can be used to detect and isolate TLT-1 proteins, regulate the bioavailability of TLT-1 proteins, and modulate TLT-1 activity.

A. Screening Assays:

The invention provides a method (also referred to herein as a “screening assay”) for identifying modulators, e.g., candidate or test compounds or agents (e.g., peptides, peptidomimetics, small molecules or other drugs) which bind to TLT-1 proteins, have a stimulatory or inhibitory effect on, for example, TLT-1 expression or TLT-1 activity, or have a stimulatory or inhibitory effect on, for example, the expression or activity of TLT-1 ligand or target molecule.

In one embodiment, the invention provides assays for screening candidate or test compounds which are target molecules of a TLT-1 protein or polypeptide or biologically active portion thereof. In another embodiment, the invention provides assays for screening candidate or test compounds which bind to or modulate the activity of a TLT-1 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses a TLT-1 protein or biologically active portion thereof is contacted with a test compound and the ability of the test compound to modulate TLT-1 activity is determined. Determining the ability of the test compound to modulate TLT-1 activity can be accomplished by monitoring, for example, the production of one or more specific metabolites in a cell which expresses TLT-1 (see, e.g., Saada et al. (2000) Biochem. Biophys. Res. Commun. 269: 382-386). The cell, for example, can be of mammalian origin, e.g., a platelet or a megakaryocyte cell.

In one embodiment, TLT-1 activity can be measured using a platelet aggregation assay or a platelet shape-change assay, as described in Jantzen, H.-M. et al. (2001) J. Clin. Invest. 108(3):477-83; Leo, L. et al. (2002) Blood 100(8):2839-44; or Nieswandt et al. (2001) J. Exp. Med. 193:459-469. Other assays that may be used to measure TLT-1 activity (e.g., as related to platelet activity) include a bleeding time assay and a platelet static adhesion assay, both described in Nieswandt, B. et al. (2001) EMBO J. 20(9):2120-30.

TLT-1 activity can also be measured using assays that measure platelet production. Reticulated platelets are young platelets that contain residual mRNA and rRNA when they are released from the bone marrow into the peripheral circulation as a result of thrombopoiesis. The level of reticulated platelets in the blood reflects the level of thrombopoiesis. Quantitation of reticulated platelets is achieved by combining a monoclonal antibody specific for platelets and an RNA-specific vital dye (thiazole orange) with analysis by flow cytometry, allowing the rapid and accurate cytometric quantitation of hundreds of events. Platelets are specifically identified for the direct determination of RNA content regardless of size and concentration. Assays for the measurement of reticulated platelets can be found, for example, in Kienast J. and Schmitz, G. (1990) Blood 75:116-121; Ault, K. A. et al. (1992) Am. J. Clin. Pathol. 98:637-646; Ault, K. A. (1993) Ann. N.Y. Acad. Sci. 677:293-308; Rinder, H. M., et.al. (1993) Arch. Pathol. Lab. Med. 117:606-610; Richards, E. and Baglin, P. (1995) Br. J. Hem. 91:445-51; and Ogata H. (1998) Kurume Med. J. 45:165-170.

TLT-1 activity can also be measured by detecting the level of ADP and/or thromboxane A2 secretion from platelets. Thromboxane A2 and ADP provide autocrine, co-stimulatory signals to support collagen-induced activation of platelets. ADP secretion is assayed by using luciferase to measure secreted ATP from platelet dense granules. The assay involves the direct addition of luciferase-luciferin to cuvettes of activated platelets followed by measurement of luminescence intensity. Thromboxane A2 is assayed by determining the concentration of thromboxane B2, a stable metabolite of thromboxane A2, in the supernatant of cultures of stimulated platelets using a commercially available ELISA kit. (See Jin et al (2002) Blood 99:193-198; and Fitzgerald (1991) Am. J. Cardiol. 68:11b-15b).

Many other assays for platelet function are known in the art (and often commercially available), all of which are contemplated for use in the methods of the invention.

TLT-1 activity can still further be measured by detecting the subcellular localization of TLT-1 (e.g., in the alpha granules and/or on the cell surface), using standard methods such as those described herein.

The ability of the test compound to modulate TLT-1 binding to a target molecule (e.g., a ligand or an intracellular signaling molecule) or to bind to another TLT-1 molecule can also be determined. Determining the ability of the test compound to modulate TLT-1 binding to a target molecule can be accomplished, for example, by coupling the TLT-1 target molecule with a radioisotope or enzymatic label such that binding of the TLT-1 target molecule to TLT-1 can be determined by detecting the labeled TLT-1 target molecule in a complex. Alternatively, TLT-1 could be coupled with a radioisotope or enzymatic label to monitor the ability of a test compound to modulate TLT-1 binding to a TLT-1 target molecule in a complex. Determining the ability of the test compound to bind TLT-1 can be accomplished, for example, by coupling the compound with a radioisotope or enzymatic label such that binding of the compound to TLT-1 can be determined by detecting the labeled TLT-1 compound in a complex. For example, compounds (e.g., TLT-1 target molecules) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a TLT-1 target molecule) to interact with TLT-1 without the labeling of any of the interactants. For example, a microphysiometer can be used to detect the interaction of a compound with TLT-1 without the labeling of either the compound or the TLT-1. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TLT-1.

In another embodiment, an assay is a cell-based assay comprising contacting a cell expressing a TLT-1. target molecule (e.g., a TLT-1 ligand) with a test compound and determining the ability of the test compound to modulate (e.g., stimulate or inhibit) the activity of the TLT-1 target molecule. Determining the ability of the test compound to modulate the activity of a TLT-1 target molecule can be accomplished, for example, by determining the ability of the TLT-1 protein to bind to or interact with the TLT-1 target molecule.

Determining the ability of the TLT-1 protein, or a biologically active fragment thereof, to bind to or interact with a TLT-1 target molecule can be accomplished by one of the methods described above for determining direct binding. In a preferred embodiment, determining the ability of the TLT-1 protein to bind to or interact with a TLT-1 target molecule can be accomplished by determining the activity of the target molecule. For example, the activity of the target molecule can be determined by detecting induction of a cellular response (i.e., cellular proliferation), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a TLT-1 protein or biologically active portion thereof is contacted with a test compound and the ability of the test compound to bind to the TLT-1 protein or biologically active portion thereof is determined. Preferred biologically active portions of the TLT-1 proteins to be used in assays of the present invention include fragments which participate in interactions with TLT-1 or non-TLT-1 molecules, e.g., fragments with high surface probability scores. Binding of the test compound to the TLT-1 protein can be determined either directly or indirectly as described above. In a preferred embodiment, the assay includes contacting the TLT-1 protein or biologically active portion thereof with a known compound which binds TLT-1 to form an assay mixture, contacting the assay mixture with a test compound, and determining the ability of the test compound to interact with a TLT-1 protein, wherein determining the ability of the test compound to interact with a TLT-1 protein comprises determining the ability of the test compound to preferentially bind to TLT-1 or biologically active portion thereof as compared to the known compound.

In another embodiment, the assay is a cell-free assay in which a TLT-1 protein or biologically active portion thereof is contacted with a test compound and the ability of the test compound to modulate (e.g., stimulate or inhibit) the activity of the TLT-1 protein or biologically active portion thereof is determined. Determining the ability of the test compound to modulate the activity of a TLT-1 protein can be accomplished, for example, by determining the ability of the TLT-1 protein to bind to a TLT-1 target molecule by one of the methods described above for determining direct binding. Determining the ability of the TLT-1 protein to bind to a TLT-1 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a TLT-1 protein can be accomplished by determining the ability of the TLT-1 protein to further modulate the activity of a downstream effector of a TLT-1 target molecule. For example, the activity of the effector molecule on an appropriate target can be determined or the binding of the effector to an appropriate target can be determined as previously described.

In yet another embodiment, the cell-free assay involves contacting a TLT-1 protein or biologically active portion thereof with a known compound which binds the TLT-1 protein to form an assay mixture, contacting the assay mixture with a test compound, and determining the ability of the test compound to interact with the TLT-1 protein, wherein determining the ability of the test compound to interact with the TLT-1 protein comprises determining the ability of the TLT-1 protein to preferentially bind to the test compound.

In more than one embodiment of the above assay methods of the present invention, it may be desirable to immobilize either TLT-1 or its target molecule to facilitate separation of complexed from uncomplexed forms of one or both of the proteins, as well as to accommodate automation of the assay. Binding of a test compound to a TLT-1 protein, or interaction of a TLT-1 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/TLT-1 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or TLT-1 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TLT-1 binding or activity determined using standard techniques.

Other techniques for immobilizing proteins on matrices can also be used in the screening assays of the invention. For example, either a TLT-1 protein or a TLT-1 target molecule can be immobilized utilizing conjugation of biotin and streptavidin. Biotinylated TLT-1 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with TLT-1 protein or target molecules but which do not interfere with binding of the TLT-1 protein to its target molecule can be derivatized to the wells of the plate, and unbound target or TLT-1 protein trapped in the wells by antibody conjugation. Methods for detecting such complexes, in addition to those described above for the GST-immobilized complexes, include immunodetection of complexes using antibodies reactive with the TLT-1 protein or target molecule, as well as enzyme-linked assays which rely on detecting an enzymatic activity associated with the TLT-1 protein or target molecule.

In another embodiment, modulators of TLT-1 expression are identified in a method wherein a cell is contacted with a candidate compound and the expression of TLT-1 mRNA or protein in the cell is determined. The level of expression of TLT-1 mRNA or protein in the presence of the candidate compound is compared to the level of expression of TLT-1 mRNA or protein in the absence of the candidate compound. The candidate compound can then be identified as a modulator of TLT-1 expression based on this comparison. For example, when expression of TLT-1 mRNA or protein is greater (statistically significantly greater) in the presence of the candidate compound than in its absence, the candidate compound is identified as a stimulator of TLT-1 mRNA or protein expression. Alternatively, when expression of TLT-1 mRNA or protein is less (statistically significantly less) in the presence of the candidate compound than in its absence, the candidate compound is identified as an inhibitor of TLT-1 mRNA or protein expression. The level of TLT-1 mRNA or protein expression in the cells can be determined by methods described herein for detecting TLT-1 ImRNA or protein.

In yet another aspect of the invention, the TLT-1 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with TLT-1 (“TLT-1-binding proteins” or “TLT-1-bp”) and are involved in TLT-1 activity. Such TLT-1-binding proteins are also likely to be involved in the propagation of signals by the TLT-1 proteins or TLT-1 targets as, for example, downstream elements of a TLT-1-mediated signaling pathway. Alternatively, such TLT-L1-binding proteins are likely to be TLT-1 inhibitors.

The two-hybrid system is based on the modular nature of most transcription factors, which consist of separable DNA-binding and activation domains. Briefly, the assay utilizes two different DNA constructs. In one construct, the gene that codes for a TLT-1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TLT-1-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TLT-1 protein. In a preferred embodiment, the TLT-1 protein used as the bait is a cytoplasmic domain. In another preferred embodiment, the TLT-1 protein used as the bait is an extracellular domain.

In another embodiment, TLT-1 modulators and/or target molecules may be identified using a chimeric receptor assay. In one example of such an assay, the TLT-1 extracellular domain and transmembrane domain (or a heterologous transmembrane domain) is fused to a heterologous cytoplasmic signaling domain that activates a detectable reporter gene. The fusion protein is expressed in cells that contain the reporter gene. The cells are then contacted with test compounds, and test compounds or target molecules that activate the reporter gene are identified as TLT-1 target molecules. The test compounds may be small molecules, peptides, or peptidomimetics, or may be polypeptides. Test compounds which are polypeptides may be soluble or expressed on the surface of a cell. In another embodiment the fusion protein comprises the TLT-1 cytoplasmic domain and transmembrane domain (or a heterologous transmembrane domain) fused to a heterologous extracellular (e.g., ligand binding) domain that responds to a known ligand (for example, the EGF ligand binding domain). The fusion protein is expressed in cells, and the cells are contacted with the ligand that binds to the heterologous ligand-binding domain (e.g., EGF). The cells can be analyzed to determine what intracellular proteins interact with the TLT-1 cytoplasmic domain, what proteins are phosphorylated, etc. in response to the ligand binding. The cells can also be transfected with a library of polypeptides (e.g., a platelet-specific library) to identify target molecules in response to ligand binding.

In another aspect, the invention pertains to a combination of two or more of the assays described herein. For example, a modulating agent can be identified using a cell-based or a cell free assay, and the ability of the agent to modulate the activity of a TLT-1 protein can be confirmed in vivo, e.g., in an animal such as an animal model for a bleeding or clotting disorder, for example, mice lacking the transcription factor NF-E2 (Lecine, P. et al. (1998) Blood 92(5):1608-1616; Lecine, P. et al. (2000) Blood 96(4):1366-1373); mice lacking a blood clotting factor such as Factor VIII or IX; the gunmetal strain of mice which shows thrombocytopenia (Swank, R. T. et al. (1993) Blood 81:2626-2635); mice with mutations in a nonmnuscle myosin heavy chain gene (Kelley, M. J. et al. (2000) Nat. Genet. 26:106-108; Consortium TM-HFS (2000) Nat. Genet. 26:103-105; Kunishima, S. et al. (2001) Blood 97:1147-1149); mice with mutations in GATA-1; mice with “mocha” and/or “pearl” mutations (Kantheti, P. et al. (1998) Neuron 21:111-122; Zhen, L. et al. (1999) Blood 94:146-155); Beige mice (Barbosa, M. D. et al. (1996) Nature 382:262-265); pallid mice (Huang, L. et al. (1999) Nat. Genet. 23:329-332); mice with mutations in thrombopoietin (TPO); mice with mutations in c-Mpl, the surface receptor for TPO (Ihara, K. et al. (1999) Proc. Natl. Acad. Sci. USA 96:3132-3136; van den Oudenrijn, S. et al. (2000) Br. J. Haematol. 110:441-448; Ballmaier, M. et al. (2001) Blood 97:139-146); and mice which over express or have mutations in HoxA11 (Thorsteinsdottir, U. et al. (1997) Mol. Cell. Biol. 17:495-505; Thompson, A. A. and Nguyen, L. T. (2000) Nat. Genet. 26:397-398); as well as animal models for stroke, heart disease, and other platelet-associated disorders.

This invention further pertains to novel agents identified by the above-described screening assays. Accordingly, it is within the scope of this invention to further use an agent identified as described herein in an appropriate animal model, as described above. For example, an agent identified as described herein (e.g., a TLT-1 modulating agent, an antisense TLT-1 nucleic acid molecule, a TLT-1-specific antibody, or a TLT-1-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections below.

A. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining TLT-1 protein and/or nucleic acid expression as well as TLT-1 activity, in the context of a biological sample (e.g., blood, serum, cells, tissue) to thereby determine whether an individual is afflicted with a disease or disorder, or is at risk of developing a disorder, associated with aberrant or unwanted TLT-1 expression or activity. The invention also provides for prognostic (or predictive) assays for determining whether an individual is at risk of developing a disorder associated with TLT-1 protein, nucleic acid expression or activity. For example, mutations in a TLT-1 gene can be assayed in a biological sample. Such assays can be used for prognostic or predictive purpose to thereby prophylactically treat an individual prior to the onset of a disorder characterized by or associated with TLT-1 protein, nucleic acid expression or activity.

Another aspect of the invention pertains to monitoring the influence of agents (e.g., drugs, compounds) on the expression or activity of TLT-1 in clinical trials.

These and other agents are described in further detail in the following sections.

1. Diagnostic Assays

An exemplary method for detecting the presence or absence of TLT-1 protein or nucleic acid in a biological sample involves obtaining a biological sample from a test subject and contacting the biological sample with a compound or an agent capable of detecting TLT-1 protein or nucleic acid (e.g., mRNA, or genomic DNA) that encodes TLT-1 protein such that the presence of TLT-1 protein or nucleic acid is detected in the biological sample. A preferred agent for detecting TLT-1 mRNA or genomic DNA is a labeled nucleic acid probe capable of hybridizing to TLT-1 mRNA or genomic DNA. The nucleic acid probe can be, for example, the TLT-1 nucleic acid set forth in SEQ ID NO:1, 3, 4, 6, 18, 20, 21, 23, 24, or 26, or a portion thereof, such as an oligonucleotide of at least 15, 30, 50, 100, 250 or 500 nucleotides in length and sufficient to specifically hybridize under stringent conditions to TLT-1 mRNA or genomic DNA. Other suitable probes for use in the diagnostic assays of the invention are described herein.

A preferred agent for detecting TLT-1 protein is an antibody capable of binding to TLT-1 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect TLT-1 ImRNA, protein, or genomic DNA in a biological sample in vitro as well as in vivo. For example, in vitro techniques for detection of TLT-1 mRNA include Northern hybridizations and in situ hybridizations. In vitro techniques for detection of TLT-1 protein include enzyme linked immunosorbent assays (ELISAs), Western blots, immunoprecipitations and immunofluorescence. In vitro techniques for detection of TLT-1 genomic DNA include Southern hybridizations. Furthermore, in vivo techniques for detection of TLT-1 protein include introducing into a subject a labeled anti-TLT-1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting TLT-1 protein, mRNA, or genomic DNA, such that the presence of TLT-1 protein, mRNA or genomic DNA is detected in the biological sample, and comparing the presence of TLT-1 protein, mRNA or genomic DNA in the control sample with the presence of TLT-1 protein, mRNA or genomic DNA in the test sample.

The invention also encompasses kits for detecting the presence of TLT-1 in a biological sample. For example, the kit can comprise a labeled compound or agent capable of detecting TLT-1 protein or mRNA in a biological sample; means for determining the amount of TLT-1 in the sample; and means for comparing the amount of TLT-1 in the sample with a standard. The compound or agent can be packaged in a suitable container. The kit can further comprise instructions for using the kit to detect TLT-1 protein or nucleic acid.

2. Prognostic Assays

The diagnostic methods described herein can furthermore be utilized to identify subjects having or at risk of developing a disease or disorder associated with aberrant or unwanted TLT-1 expression or activity. As used herein, the term “aberrant” includes a TLT-1 expression or activity which deviates from the wild type TLT-1 expression or activity. Aberrant expression or activity includes increased or decreased expression or activity, as well as expression or activity which does not follow the wild type developmental pattern of expression or the subcellular pattern of expression. For example, aberrant TLT-1 expression or activity is intended to include the cases in which a mutation in the TLT-1 gene causes the TLT-1 gene to be under-expressed or over-expressed and situations in which such mutations result in a non-functional TLT-1 protein or a protein which does not function in a wild-type fashion, e.g., a protein which does not interact with a TLT-1 target molecule, or one which interacts with a non-TLT-1 target molecule. As used herein, the term “unwanted” includes an unwanted phenomenon involved in a biological response such as bleeding or clotting. For example, the term unwanted includes a TLT-1 expression or activity which is undesirable in a subject.

The assays described herein, such as the preceding diagnostic assays or the following assays, can be utilized to identify a subject having or at risk of developing a disorder associated with a misregulation in TLT-1 protein activity or nucleic acid expression, such as a platelet-associated disorder. Alternatively, the prognostic assays can be utilized to identify a subject having or at risk for developing a disorder associated with a misregulation in TLT-1 protein activity or nucleic acid expression, such as a platelet-associate disorder. Thus, the present invention provides a method for identifying a disease or disorder associated with aberrant or unwanted TLT-1 expression or activity in which a test sample is obtained from a subject and TLT-1 protein or nucleic acid (e.g., mRNA or genomic DNA) is detected, wherein the presence of TLT-1 protein or nucleic acid is diagnostic for a subject having or at risk of developing a disease or disorder associated with aberrant or unwanted TLT-1 expression or activity. As used herein, a “test sample” refers to a biological sample obtained from a subject of interest. For example, a test sample can be a biological fluid (e.g., cerebrospinal fluid or serum), cell sample, or tissue sample (e.g., a bone marrow sample).

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TLT-1 expression or activity. For example, such methods can be used to determine whether a subject can be effectively treated with an agent for a platelet associated disorder. Thus, the present invention provides methods for determining whether a subject can be effectively treated with an agent for a disorder associated with aberrant or unwanted TLT-1 expression or activity in which a test sample is obtained and TLT-1 protein or nucleic acid expression or activity is detected (e.g., wherein the abundance of TLT-1 protein or nucleic acid expression or activity is diagnostic for a subject that can be administered the agent to treat a disorder associated with aberrant or unwanted TLT-1 expression or activity).

The methods of the invention can also be used to detect genetic alterations in a TLT-1 gene, thereby determining if a subject with the altered gene is at risk for a disorder characterized by misregulation in TLT-1 protein activity or nucleic acid expression, such as a platelet associated disorder. In preferred embodiments, the methods include detecting, in a sample of cells from the subject, the presence or absence of a genetic alteration characterized by at least one of an alteration affecting the integrity of a gene encoding a TLT-1-protein, or the mis-expression of the TLT-1 gene. For example, such genetic alterations can be detected by ascertaining the existence of at least one of 1) a deletion of one or more nucleotides from a TLT-1 gene; 2) an addition of one or more nucleotides to a TLT-1 gene; 3) a substitution of one or more nucleotides of a TLT-1 gene, 4) a chromosomal rearrangement of a TLT-1 gene; 5) an alteration in the level of a messenger RNA transcript of a TLT-1 gene, 6) aberrant modification of a TLT-1 gene, such as of the methylation pattern of the genomic DNA, 7) the presence of a non-wild type splicing pattern of a messenger RNA transcript of a TLT-1 gene, 8) a non-wild type level of a TLT-1-protein, 9) allelic loss of a TLT-1 gene, and 10) inappropriate post-translational modification of a TLT-1-protein. As described herein, there are a large number of assays known in the art which can be used for detecting alterations in a TLT-1 gene. A preferred biological sample is a tissue (e.g., a bone marrow sample) or blood sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al (1988) Science 241:1077-1080; and Nakazawa et al. (1994) Proc. Natl. Acad. Sci. USA 91:360-364), the latter of which can be particularly useful for detecting point mutations in a TLT-1 gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a TLT-1 gene under conditions such that hybridization and amplification of the TLT-1 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a TLT-1 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in TLT-1 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nat. Med. 2:753-759). For example, genetic mutations in TLT-1 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the TLT-1 gene and detect mutations by comparing the sequence of the sample TLT-1 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al (1996) Adv. Chromatogr. 36:127-162; and Griffin et al. (1993) Appl. Biochem. Biotechnol. 38:147-159).

Other methods for detecting mutations in the TLT-1 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type TLT-1 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in TLT-1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a TLT-1 sequence, e.g., a wild-type TLT-1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in TLT-1 genes. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat. Res. 285:125-144; and Hayashi (1992) Genet. Anal. Tech. Appl. 9:73-79). Single-stranded DNA fragments of sample and control TLT-1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a TLT-1 gene.

Furthermore, any cell type or tissue in which TLT-1 is expressed may be utilized in the prognostic assays described herein.

3. Monitoring of Effects During Clinical Trials

Monitoring the influence of agents (e.g., drugs) on the expression or activity of a TLT-1 protein (e.g., on the modulation of platelet activity) can be applied not only in basic drug screening, but also in clinical trials. For example, the effectiveness of an agent determined by a screening assay as described herein to increase TLT-1 gene expression, protein levels, or upregulate TLT-1 activity, can be monitored in clinical trials of subjects exhibiting decreased TLT-1 gene expression, protein levels, or downregulated TLT-1 activity. Alternatively, the effectiveness of an agent determined by a screening assay to decrease TLT-1 gene expression, protein levels, or downregulate TLT-1 activity, can be monitored in clinical trials of subjects exhibiting increased TLT-1 gene expression, protein levels, or upregulated TLT-1 activity. In such clinical trials, the expression or activity of a TLT-1 gene, and preferably, other genes that have been implicated in, for example, a TLT-1-associated disorder can be used as a “read out” or markers of the phenotype of a particular cell.

For example, and not by way of limitation, genes, including TLT-1, that are modulated in cells by treatment with an agent (e.g., compound, drug or small molecule) which modulates TLT-1 activity (e.g., identified in a screening assay as described herein) can be identified. Thus, to study the effect of agents on TLT-1-associated disorders (e.g., disorders characterized by deregulated bleeding, clotting, or platelet activity), for example, in a clinical trial, cells can be isolated and RNA prepared and analyzed for the levels of expression of TLT-1 and other genes implicated in the TLT-1-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of TLT-1 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a TLT-1 protein, mRNA, or genomic DNA in the preadministration sample; (iii) obtaining one or more post-administration samples from the subject; (iv) detecting the level of expression or activity of the TLT-1 protein, mRNA, or genomic DNA in the post-administration samples; (v) comparing the level of expression or activity of the TLT-1 protein, mRNA, or genomic DNA in the pre-administration sample with the TLT-1 protein, mRNA, or genomic DNA in the post administration sample or samples; and (vi) altering the administration of the agent to the subject accordingly. For example, increased administration of the agent may be desirable to increase the expression or activity of TLT-1 to higher levels than detected, i.e., to increase the effectiveness of the agent. Alternatively, decreased administration of the agent may be desirable to decrease expression or activity of TLT-1 to lower levels than detected, i.e., to decrease the effectiveness of the agent. According to such an embodiment, TLT-1 expression or activity may be used as an indicator of the effectiveness of an agent, even in the absence of an observable phenotypic response.

B. Methods of Treatment:

The present invention provides for both prophylactic and therapeutic methods of treating a subject at risk of (or susceptible to) a disorder or having a disorder associated with aberrant or unwanted TLT-1 expression or activity, e.g., a platelet-associated disorder, as described elsewhere herein. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

1. Prophylactic Methods

In one aspect, the invention provides a method for preventing in a subject, a disease or condition associated with an aberrant or unwanted TLT-1 expression or activity, by administering to the subject a TLT-1 or an agent which modulates TLT-1 expression or at least one TLT-1 activity: Subjects at risk for a disease which is caused or contributed to by aberrant or unwanted TLT-1 expression or activity can be identified by, for example, any or a combination of diagnostic or prognostic assays as described herein. Administration of a prophylactic agent can occur prior to the manifestation of symptoms characteristic of the TLT-1 aberrancy, such that a disease or disorder is prevented or, alternatively, delayed in its progression. Depending on the type of TLT-1 aberrancy, for example, a TLT-1, TLT-1 agonist or TLT-1 antagonist agent can be used for treating the subject. The appropriate agent can be determined based on screening assays described herein.

2. Therapeutic Methods

Another aspect of the invention pertains to methods of modulating TLT-1 expression or activity for therapeutic purposes. Accordingly, in an exemplary embodiment, the modulatory method of the invention involves contacting a cell with a TLT-1 or agent that modulates one or more of the activities of TLT-1 protein activity associated with the cell. An agent that modulates TLT-1 protein activity can be an agent as described herein, such as a nucleic acid or a protein, a naturally-occurring target molecule of a TLT-1 protein (e.g., a TLT-1 ligand), a TLT-1 antibody, a TLT-1 agonist or antagonist, a peptidomimetic of a TLT-1 agonist or antagonist, or other small molecule. In one embodiment, the agent stimulates one or more TLT-1 activities. Examples of such stimulatory agents include active TLT-1 protein and a nucleic acid molecule encoding TLT-1 that has been introduced into the cell. In another embodiment, the agent inhibits one or more TLT-1 activities. Examples of such inhibitory agents include antisense TLT-1 nucleic acid molecules, anti-TLT-1 antibodies, and TLT-1 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a TLT-1 protein or nucleic acid molecule. In one embodiment, the method involves administering an agent (e.g., an agent identified by a screening assay described herein), or combination of agents that modulates (e.g., upregulates or downregulates) TLT-1 expression or activity. In another embodiment, the method involves administering a TLT-1 protein or nucleic acid molecule as therapy to compensate for reduced, aberrant, or unwanted TLT-1 expression or activity.

Stimulation of TLT-1 activity is desirable in situations in which TLT-1 is abnormally downregulated and/or in which increased TLT-1 activity is likely to have a beneficial effect. Likewise, inhibition of TLT-1 activity is desirable in situations in which TLT-1 is abnormally upregulated and/or in which decreased TLT-1 activity is likely to have a beneficial effect.

This invention is further illustrated by the following examples which should not be construed as limiting. The contents of all references, patents, and published patent applications cited throughout this application, as well as the figures and the sequence listing, are incorporated herein by reference.

### EXAMPLES

Materials and Methods

The following materials and methods were used in Examples 1-5

Sequence Information

Sequence information and genomic structural characteristics were determined using the Celera Discovery System. In some cases public information was used and the sequences confirmed by the Celera Discovery System. The validity of both the TREM-like and non-TREM-like murine sequences of the cluster was confirmed using reverse transcription-polymerase chain reaction (RT-PCR) and the following primer pairs: TREM 1: 5′-gagcttgaaggatgaggaag-3′ (SEQ ID NO:7), 5′-gctcctcctgtgaaatagac-3′ (SEQ ID NO:8); TREM 2: 5′-cccaagcttaacaccacggtgctgcagg-3′ (SEQ ID NO:9), 5′-cgcggatcctgactggacttaagctgta-3′ (SEQ ID NO:10); TREM 3: 5′-gttagcacaccaggaaggag-3′ (SEQ ID NO:11), 5′-ctgtttctcagagactccctg-3′ (SEQ ID NO:12); FJL13693: 5′-atggatggatttgtcacgac-3′ (SEQ ID NO:13), 5′-aaatccagccatcatcacag-3′ (SEQ ID NO:14).

Epitope Tagging

Full-length murine TLT-1 (TREM-like transcript-1; GenBank accession number AY078502; SEQ ID NO:1) was generated by RT-PCR using Platinum HiFi-Supermix (Gibco-BRL, Grand Island, N.Y.) with bone marrow cDNA as a template. The resulting cDNA was epitope tagged using the TOPO V5 epitope tag system from Invitrogen (Carlsbad, Calif.). The primers used were 5′-agaacctactactgcccag-3′ (SEQ ID NO:15) and 5′-gccaatatgtaatgacggtag-3′ (SEQ ID NO:16).

Tissue and Cell Line Expression

The expression of TLT-1 in normal tissue and cell lines was determined using RT-PCR. Total RNA was made using Trizol (Gibco-BRL) according to the manufacturer's instructions. First-strand synthesis was achieved using the Superscript cDNA synthesis kit (Gibco-BRL). PCR cycles were as follows: 95° C. for 2 minutes; 30 cycles of 94° C. for 30 sec, 55° C. for 30 sec, and 72° C. and 1 minute. For Northern analysis, 30 pg of RNA was used per lane according to the methods described in Musso, T. et al. (1995) J. Exp. Med. 181:1425-1431.

Transfections and Immunoprecipitation

Phosphorylation and protein-protein interactions were analyzed using HEK293T cells as described in Paul, S. P. et al. (2000) Blood 96:483-490. Expression plasmids for TLT-1 were described in Washington, A. V. et al. (2002) Blood 100:3822-3824.

Cell Purifications and Stimulation

Mice were bred and maintained under specific pathogen free conditions at the NCI-Frederick. Peripheral blood collection via cardiac puncture of anesthetized mice was carried out as described. Purified platelets were isolated from peripheral blood(as described in Jantzen, H. M. et al. (2001) J. Clin. Invest. 108:477-483), and washed in modified Tyrodes buffer (10 mM HEPES pH 7.4, 138 mM NaCl, 5.5 mM glucose, 2.9 mM KCl, and 12 mM NaHCO₃). For stimulation cells were resuspended in modified Tyrodes at a density of 2×10⁸/ml. EGTA was added to a final concentration of 5 mM to prevent clumping and cells were stimulated with 2-5 U/ml purified human Thrombin (Calbiochem, La Jolla, Calif.) at 37° for 10 min. Stimulation was stopped by centrifugation onto poly-L-lysine coated slides. Murine bone marrow MK were enriched through culture of unfractionated bone marrow with recombinant murine thrombopoietin (Calbiochem, San Diego, Calif., 3 ng/ml) in RPMI-1640 supplemented with 2 mM Glutamine, 10% fetal bovine serum, and antibiotics for 7-10 days. After culture, cells were collected, washed, and cytospun onto poly-L-lysine coated slides. Murine dendritic cells were generated through culture of unfractionated bone marrow in RPMI-1640 supplemented with 2 mM Glutamine, 10% fetal bovine serum, antibiotics, and recombinant IL-4 and GM-C SF (Peprotech, Rocky Hill, N.J.) for 5-7 days. Phenotype was confirmed by the expression of I-A^{b}, CD11c, and Gr-1 as detected by FACS. Human peripheral blood monocytes and platelets were isolated by elutriation from the blood of healthy donors (as-described in Musso, T. et al. (1994) J. Exp. Med. 180:2383-2388).

Antibodies

Biotin conjugated anti-CD11c, biotin-conjugated anti-CD62P, FITC-conjugated anti-I-A^{b}, APC conjugated Streptavidin and appropriate controls were from PharMingen (San Diego, Calif.). Anti murine CD62P for immunofluorescence was from R&D (Minneapolis, Minn.). Anti-TLT-1 was generated by immunizing rabbits with a fusion protein comprised of the TLT-1 Ig-domain (FusionAntibodies Inc, Belfast, Northern Ireland). For FACS, anti-TLT-1 was detected using FITC-conjugated goat anti-rabbit IgG (Jackson Immunoresearch, West Grove, Pa.).

Confocal Microscopy

Cytospun platelets or bone marrow samples were fixed using BD Cytofix/Cytoperm, then blocked in 1×BD Perm/Wash (BD Biosciences, San Diego Calif.). Primary antibody was diluted in Perm/Wash. After antibody incubations, slides were washed in Perm/Wash. Anti-murine TLT-1 and anti-murine CD62P were visualized without bleed-through by using Alexa Fluor 488 goat anti-rabbit IgG and Alexa Fluor 633 donkey anti-goat IgG (Molecular Probes, Eugene Oreg.), respectively. Controls included slides stained with either primary antibody alone and counterstained with both secondary antibodies demonstrating that the secondary reagents do not react with one another. Additional controls included slides stained with secondaries without primaries, and slides stained with irrelevant primary antibodies. Cells were examined using a Zeiss LSM 510 NLO Inverted Confocal Laser Scanning Microscope equipped with a Plan-Neofluar 100×/1.3 oil objective (Carl Zeiss, Jena, Germany). Images were viewed and overlaid using the Ziess LSM Image Browser software.

### Example 1

### Identification of TLT-1

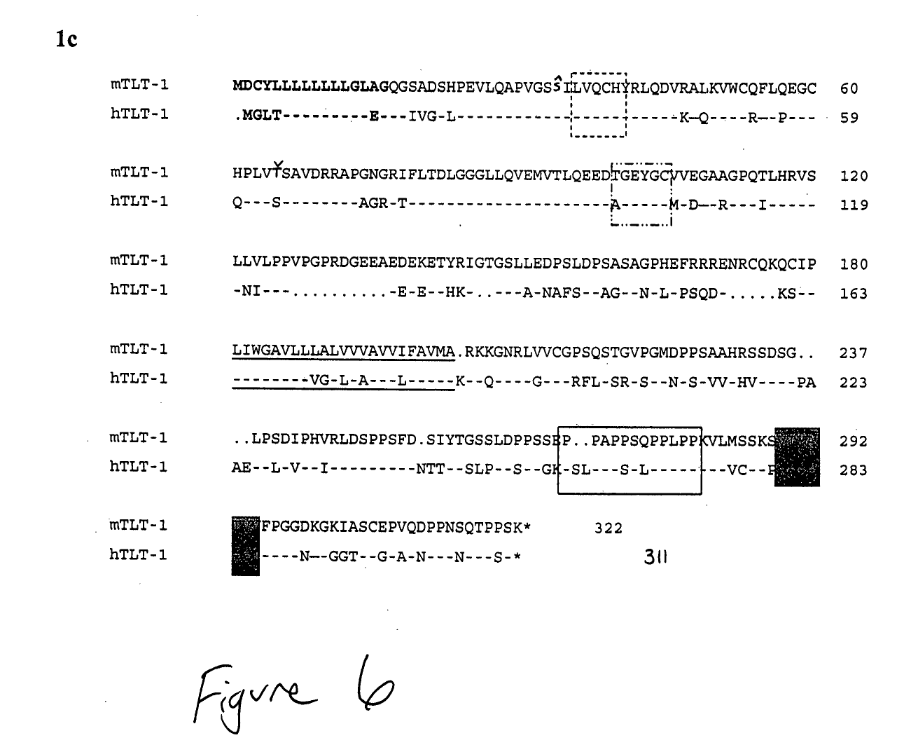

Analysis of the murine TREM cluster revealed a putative regulatory receptor referred to herein as TLT-1 (TREM-like transcript-1; set forth as SEQ ID NO:1), just telomeric to TREM 2. This cDNA encodes a single open reading frame predicting a 322-amino acid protein, set forth as SEQ ID NO:2, containing a leader sequence and a single V-set Ig domain (FIGS. 5 and 6). In stark contrast to the TREMs, the TLT-1 functional domain appears to be in the carboxy-terminus (FIG. 2) (Bouchon, A. et al. (2001) Nature 410:1103-1107). The cytoplasmic region of TLT-1 contains an immunoreceptor tyrosine-based inhibitory motif (ITIM), implying the ability to mediate inhibition through the recruitment of Src homology (SH) 2 domain-containing protein tyrosine phosphatases and/or lipid phosphatases. In addition, TLT-1 contains a polyproline-rich segment, suggesting that it has the ability to interact with SH3 domain and/or WW domain containing targets. The human TLT-1 polypeptide (SEQ ID NO:5) shows 70% identity at the amino acid level with murine TLT-1 and is similarly located within the TREM cluster (FIG. 5).

### Example 2

### Analysis of TLT-1 Expression

Given the similarities between TLT-1 and the TREMs, TLT-1 expression was examined and compared to the expression patterns of TREM 1 and 2. Preliminary screening by RT-PCR revealed TLT-1 message in murine bone marrow, brain, liver, peritoneal monocytes, P815 mastocytoma cells, and RAW264.7 macrophages. TLT-1 transcript was not seen in spleen, lung, or thymus. In RAW and dendritic cells, RT-PCR demonstrated a minor mRNA species lacking 235 bp (see FIG. 5). This mRNA predicts a truncated polypeptide with no apparent homology to the TREMs. In contrast to RT-PCR, Northern analysis demonstrated significant 1.2-kb TLT-1 mRNA only in bone marrow. The 1.2-kb mRNA confirmed that the nucleotide sequence of TLT-1 represents a full-length transcript. Similar to TLT-1, TREM 1 and 2 mRNA also was found predominantly in bone marrow, suggesting significant coexpression of TLT-1 and the activating TREM. TREM 2, but not TLT-1 or TREM 1, also is highly expressed in the RAW cells.

Further analysis revealed that TLT-1 is highly expressed in peripheral blood platelets. Flow-cytometry and western blotting using an anti-mouse-TLT-1 polyclonal antibody revealed high expression of TLT-1 in mouse platelets. Platelets were identified based on size (2-fold smaller in size on a logarithmic scale than leukocytes) and positive expression of the platelet-specific marker CD41. High expression of human TLT-1 was shown in human platelets by Northern blotting.

### Example 3

### TLT-1 is a Cell-Surface Protein

Immunoprecipitation and Western blot analysis of surface biotinylated cells expressing epitope-tagged TLT-1 confirmed TLT-1 surface expression by revealing biotin-labeled receptor of the expected molecular weight. Using nonreducing buffer, some TLT-1 protein migrates at 75 kDa, suggesting it can exist as a homodimer on the cell surface.

### Example 4

### TLT-1 Interacts with SHP-1

TLT-1 contains an ITIM and, therefore, might recruit a protein phosphatase such as SHP-1 when phosphorylated. To test this possibility, HEK293T cells were transfected with TLT-1 alone or together with SHP-1, treated with pervanadate, then immunoprecipitated with anti-V5. The resulting immunoblots were then serially probed with antiphosphotyrosine, anti-SHP-1, and then anti-V5. These experiments demonstrated that once phosphorylated, TLT-1 interacts with SHP-1. Based on these results, TLT-1 clearly has the potential for inhibition.

### Example 5

### TLT-1 is Phosphorylated by SRC but not SYK

HEK293T cells were transfected with TLT-1 alone or together with either Src (ASRC, a constitutively active form of Src) or Syk, treated with pervanadate, then immunoprecipitated with anti-V5. The resulting immunoblots were then serially probed with antiphosphotyrosine and then anti-V5. These experiments demonstrated that TLT-1 is phosphorylated by Src but not Syk.

### Example 6

### Production of TLT-1 Specific Antibodies and Fusion Proteins

A polyclonal antibody to the mouse TLT-1 protein has been produced that specifically recognizes mouse TLT-1 by Western blot and flow cytometry. The antibody was produced by Fusion Antibodies (Belfast, Northern Ireland). Rabbits were immunized with a fusion protein comprising a portion of the extracellular domain of mouse TLT-1 (amino acid residues 21-168 of SEQ ID NO:2) to a poly-histidine tag.

An antibody to the human TLT-1 was generated in rabbits by immunization with a peptide that is highly conserved in the c-terminus of both mouse and human TLT-1 (CDVPHIRLDSPPSFDN; set forth as SEQ ID NO:17), which has only two differences between mouse and human. The peptide corresponds to amino acid residues 228-242 of SEQ ID NO:5, with the addition of a cysteine residue at the N-terminus. The antibody can detect both mouse and human TLT-1, and indicates that both mouse and human TLT-1 is localized to platelet granules (see below).

Monoclonal antibodies are being produced by a number of methods, including immunization of rats with mouse platelets and/or cells transfected with TLT-1 cDNA and/or the Fc-fusion protein of the extracellular domain. The Fc-TLT-1 fusion was produced using the methods of Winter, C. C. and Long, E. O. (2000) Methods Mol. Biol. 121:239-50. The fusion contains a portion of the mouse TLT-1 extracellular domain (amino acid residues 20-171 of SEQ ID NO:2) fused to the Fc region of human IgG.

### Example 7

### Further Analysis of TLT-1 Expression

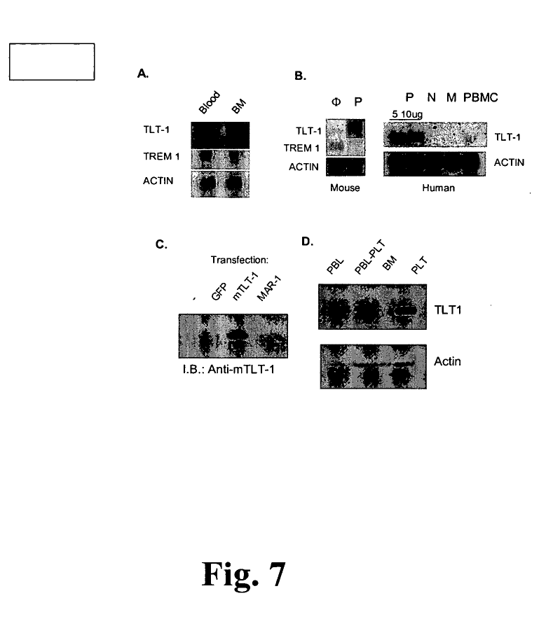

To identify TLT-1 expressing leukocytes in the periphery, RNA was extracted from whole blood. This analysis demonstrated an extraordinary level of TLT-1 message relative to bone marrow (FIG. 7A). The high levels of TLT-1 mnRNA in peripheral blood, together with the lack of TLT-1 mRNA in thymus, and lymph node, suggested TLT-1 might be expressed only within peripheral blood platelets and their bone marrow precursors. This possibility was tested by comparing TLT-1 and TREM-1 mRNA levels in murine bone marrow derived macrophages and purified platelets. TLT-1 was highly expressed in platelets whereas TREM-1 was detectable only in macrophages. This strikingly limited expression pattern of TLT-1 was further confirmed by comparison of human peripheral blood mononuclear cells (PBMC), purified monocytes, purified polymorphonuclear neutrophils (PMN), and platelets by Northern analysis (FIG. 7B). TLT-1 mRNA was detectable only in platelets (FIG. 7B). This is inconsistent with the apparent high levels of TLT-1 mRNA detected in blood and purified platelets. Therefore, a polyclonal anti-TLT-1 antibody was generated against the extracellular domain of murine TLT-1. This antibody reacts well with TLT-1 expressed in HEK293T cells, but not in transfection controls or cells expressing MAR-1, a receptor with homology to TREMs and TLT-1 (FIG. 7C). Western analysis of TLT-1 in blood leukocytes (FIG. 7D) demonstrated a low level of TLT-1 (lane 1) with an apparent mass of 38-40 kDa that was eliminated when platelets were removed by slow speed centrifugation (lane 2). Unfractionated bone marrow cells (lane 3) also showed no detectable TLT-1 (lane 3) but lysate derived from enriched platelets showed readily detectable levels of TLT-1 (lane 4). Probing this filter with anti-actin confirmed near equal protein loading (FIG. 7D, lower panel). Immunohistochemistry of peripheral blood leukocytes confirmed TLT-1 expression only in platelets.

### Example 8

### Further Analysis of TLT-1 Cell-Surface Expression

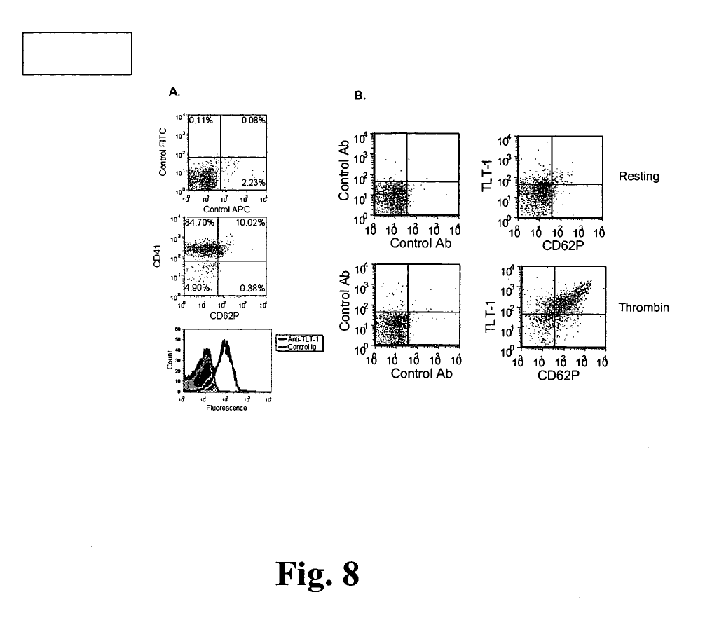

The potential for TLT-1 expression on the surface of freshly isolated murine peripheral blood platelets was next determined by flow cytometry. Platelets were identified by their light scatter properties and their expression of CD41. Their resting phenotype was confirmed by the relative lack of CD62P expression (Larsen, E. et al. (1989) Cell 59:305-312; Johnston, G. I. et al. (1989) Cell 56:1033-1044). Staining with TLT-1 revealed only a modest shift, suggesting all platelets have a low level of surface TLT-1 (FIG. 8A). Control sera showed no staining. Similarly, flow cytometric analysis of bone marrow leukocytes detected no appreciable surface TLT-1. Together these data define TLT-1 as the first known inhibitory receptor to be expressed exclusively in platelets.

FIG. 8B shows a massive upregulation of TLT-1 on the platelet surface after 10 minutes of stimulation with thrombin. Similar results were found with collagen stimulation. Moreover, upregulation of TLT-1 appeared to parallel the surface expression of CD62P over a variety of stimulation dosages and times. Together, these data suggest that TLT-1, similar to CD62P, might be sequestered within platelet granules. Detection of an agonist by the platelet would then result is rapid upregulation of both TLT-1 and CD62P.

### Example 9

### TLT-1 is Sequestered in Platelet Granules

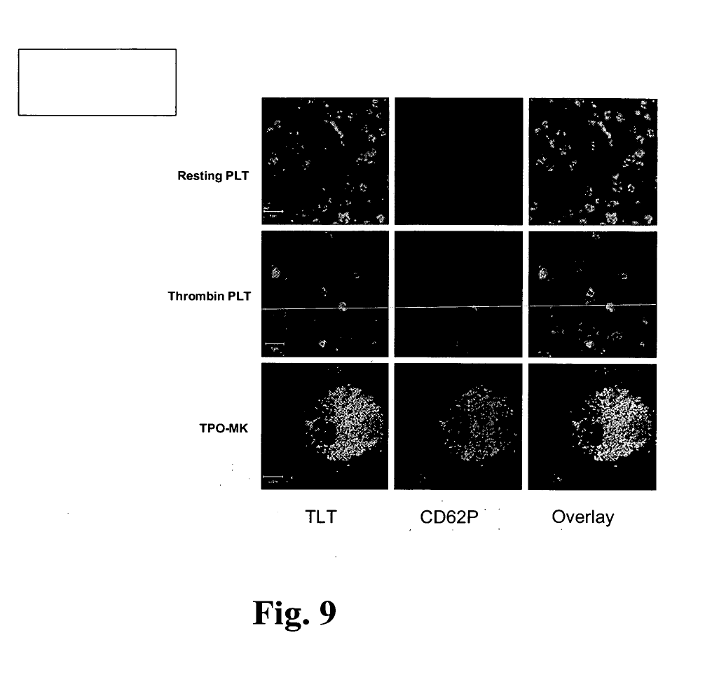

The apparent concurrent upregulation of TLT-1 and CD62P suggested that a-granules would be the most likely candidates for the intracellular pools of TLT-1. To address if TLT-1 was stored in the a-granules, resting platelets, or those first stimulated with thrombin, were fixed, permeabilized and stained with combinations of anti-TLT, anti-CD62P and analyzed using confocal microscopy. FIG. 9 shows representative confocal images of resting (top) and thrombin activated (bottom) platelets stained with anti-TLT-1 (green) and anti-CD62P (red). Note that both CD62P and TLT-1 staining exhibit relatively low background fluorescence with strong granular staining. Overlay of the images shows a high degree of co-localization, suggesting that TLT-1 is localized within the a-granule along with CD62P. The identity of TLT-1 containing granules as alpha granules was further confirmed by staining platelets from ruby mice. These mice have defective platelet dense granules due to defects in granule packaging and/or production machinery (Zhang, Q. et al. (2003) Nat. Genet. 33:145-153). There was no appreciable effect of this mutation of TLT-1 distribution.

Studies on human platelets show similar results to the murine platelets, confirming TLT-1's expression in human platelets. Upon stimulation, the majority of CD62P quickly moves to the periphery of the cell, consistent with the dramatic increase in CD62P staining by FACS demonstrated in FIG. 8B. In contrast, although the granular staining of TLT-1 is clearly gone, there is often TLT-1 protein retained in the central portion of the platelets as if demarcated by the contracted marginal band (Harrison, P. and Cramer, E. M. (1993) Blood Rev. 7:52-62, and references therein). The degree to which TLT-1 is retained in this fashion is variable, and may reflect the intensity of the activation signal and/or other parameters of the stimulation.

### Example 10

### TLT-1 is Produced and Packaged within Megakaryocytes

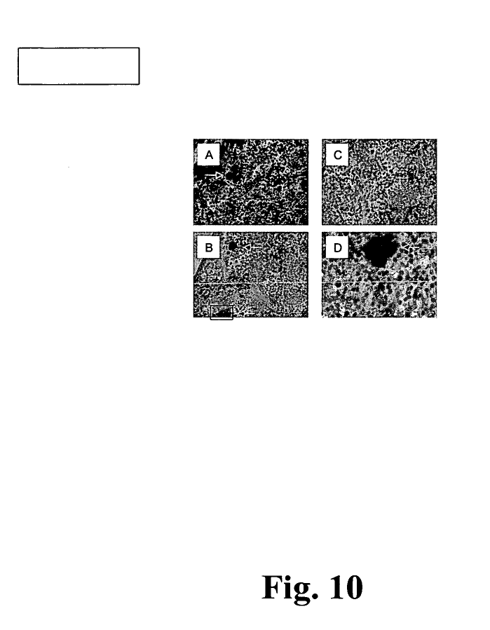

Platelets are derived from megakaryocytes (MKs); however, platelet granular content can be derived either from plasma pools via pinocytosis or endocytosis, or by production and granule packaging by either the MK or platelet (Harrison, P. and Cramer, E. M. (1993) Blood Rev. 7:52-62). lnmunohistochemisty of mouse spleen (FIGS. 10A-10D) confirmed the lineage specificity of TLT-1 and showed high levels of TLT-1 only in splenic MKs and platelets of the red pulp. Together with the levels of TLT-1 message detectable in bone marrow, this finding suggested MKs as the source of platelet TLT-1. This hypothesis was confirmed by performing confocal microscopy to localize TLT-1 and CD62P in primary bone marrow derived MKs and MKs derived via in vitro culture with TPO. Culture of unfractionated bone marrow in TPO for 7-10 days resulted in the appearance of large immature MKs. These cells are 50-75 um in diameter and contain abundant alpha granules and large multilobular nuclei but do not produce proplatelets under these culture conditions (Cramer, E. M. et al. (1997) Blood 89:2336-2346).

The results shown in FIG. 9 demonstrate that these early MKs also exhibit significant co-localization of TLT-1 and CD62P suggesting that TLT-1 has already been packaged into the α-granules at this stage of thrombopoeisis. Staining of primary bone marrow with anti-TLT-1 demonstrated large clouds of prepackaged, TLT-1-positive, granules surrounding a large multilobular nucleus further supporting a model where TLT-1 is produced and packaged within MKs.

### Example 10

### Production of Fluorescent Fusion Constructs and TLT-1 Truncation Mutants

This example describes the generation of fusion constructs containing TLT-1 and fluorescent proteins, as well as the generation of two truncation mutants of mouse TLT-1. The TLT-1 proteins were fused to the fluorescent proteins eCFP and eYPF using the TOPO expression vector pEF V5 HIS TOPO (Invitrogen, Carlbad, Calif.). The eCFP (Promega, Madison, Wis.) was isolated by PCR and blunt-end ligated to the C-terminus of the TLT-1 mutants at the vector EcoRV site.

One fusion protein contained the full-length mouse TLT-1 (SEQ ID NO:2) fused to eYFP. The truncation mutants were fused to eCFP and included either amino acid residues 1-163 or 1-248 of SEQ ID NO:2.

Taken together, these findings provide several lines of evidence showing that TLT-1 is a regulatory component of the TREM cluster. First, the homology of TLT-1 with the TREM proteins indicates a common ancestor and possibly even similar ligands. Second, the pattern of TLT-1 expression overlaps with TREM 2 and is identical to TREM 1, making them potential targets for TLT-1 mediated inhibition. Third, TLT-1 possesses the physical characteristics necessary for inhibitory signaling, most importantly, the ability to recruit SHP-1 and/or SHIP-1. The identification of an inhibitor within the TREM family adds the TREM to the growing list of paired immune receptor systems (Taylor, L. S. et al. (2000) Rev. Immunogenet. 2:204-219). Although a member of the ever-growing superfamily of inhibitory receptors, TLT-1 appears to be unique in that it contains cytoplasmic motifs for the recruitment of both SH2, SH3, and WW domain-containing proteins. The existence of a proline-rich segment in the cytoplasmic domain of a receptor is rare. This proline-rich domain may be involved in recruiting the kinases necessary to mediate phosphorylation of TLT-1. Alternatively, the proline-rich domain may be involved in bringing SH3 domain- and/or WW domain-containing phosphoproteins into proximity so they can be dephosphorylated by TLT-1 bound SHP-1 and/or SHIP-1.

Given the growing body of evidence suggesting regulation of both the innate and adaptive immune response by members of the TREM family, the finding that TLT-1 is platelet and megakaryocyte specific is surprising. The results presented herein make TLT-1 the first gene within the TREM cluster to be expressed within a single lineage. In addition, TLT-1 becomes only the second inhibitory receptor to be described in platelets, the other being PECAM (Hua, C. T. et al. (1998) J. Biol. Chem. 273:28332-28340). PECAM, however, is also expressed in endothelial cells. The analysis herein of multiple murine endothelial cell lines showed no substantial TLT-1, and immunostaining of primary murine lung endothelial cells yielded cells that were CD62P positive, and had clear von Willebrand's factor containing weibel palade bodies, but these cultures had no cells exhibiting TLT-1 staining over control values. In addition, immunohistochemistry did not detect significant levels of TLT-1 in endothelium. Taken together, this makes TLT-1 the only platelet specific inhibitory receptor described to date. Moreover, TLT-1's specificity makes it a good marker for megakaryocytes and platelets.

Platelets are highly reactive cells that carry large quantities of both soluble and cell bound cargo in two principle types of secretory granules, alpha granules and dense granules (Reviewed in Rendu, F. and Brohard-Bohn, B. (2001) Platelets 12:261-273). These granules sequester highly reactive compounds and/or receptors that are only made available when the platelet is stimulated with indicators of vascular damage (Rendu, F. and Brohard-Bohn, B. (2001) Platelets 12:261-273). Within seconds of exposure to an agonist such as thrombin or collagen platelets undergo a dramatic change in cellular morphology, including contraction of the platelet marginal band towards the center of the cell and the formation of adhesive pseudopods (Reviewed in Harrison, P. and Cramer, E. M. (1993) Blood Rev. 7:52-62; George, J. N. (2000) Lancet 355:1531-1539). Simultaneously, there is a release of granule contents resulting in dramatic increases in cell surface expression of several receptor proteins; prominent among these is the platelet selectin, CD62P and now TLT-1. The high levels of CD62P facilitate the tethered rolling required for additional platelet receptors to further interrogate the vascular wall (Rendu, F. and Brohard-Bohn, B. (2001) Platelets 12:261-273). The demonstration of co-compartmentalization and display of TLT-1 and CD62P suggests that, like CD62P, TLT-1 may play an important role in the adhesion of activated platelets to endothelium or one another. In addition, this tight regulation of the bio-availability of granule proteins like TLT-1 may explain why this work is the first to demonstrate TLT-1 in platelets despite multiple platelet proteomic studies (O'Neill, E. E. et al. (2002) Proteomics 2:288-305; Marcus, K. et al. (2000) Electrophoresis 21:2622-2636). Based on the data presented herein showing the ability of the cytoplasmic domain of TLT-1 to recruit SHP-1, TLT-1 might dampen the platelet aggregation response, or perhaps play a role in reversible platelet adhesion.

The data presented herein demonstrate that stimulation of platelets with thrombin or collagen dramatically up-regulates surface expression of both TLT-1 and CD62P; however, even when CD62P is seen to fully redistribute, TLT-1 is often retained in the central portion of the cell in a distinct ring or disc pattern. This staining pattern is reminiscent of the pattern seen when activated platelets are stained with phalloidin or tubulin suggesting that TLT-1 is retained within, or in proximity of, the contracting marginal band (Italiano, J. E. et al. (2003) Blood 101:4789-4796). In light of this difference in subcellular location post-activation, it is interesting to note that although both TLT-1 and CD62P are both packaged into alpha granules by the MK sorting machinery, the cytoplasmic tails of the two receptors vary greatly. Whereas the cytoplasmic domain of CD62P is only 35 residues, perhaps just enough to target it to the granule and anchor the receptor for its role as an adhesion molecule, that of TLT-1 is 118 residues long and contains multiple potential protein-protein interaction domains. The difference in distribution may possibly reflect a more prominent role for TLT-1 in the production, movement, or regulation of granule function, or may serve strictly as granule cargo, as is the case for CD62P. Interestingly, studies utilizing the pearl mutation affecting AP3 function have suggested the existence of myeloid lineage specific machinery for packaging granule cargo such as CD62P (Daugherty, B. L. et al. (2001) Traffic 2:406-413). It is possible that the extensive cytoplasmic tail of TLT-1 may be a reflection of a role for TLT-1 in MK specific granule formation and sorting.

In addition to packaging proteins produced in the MK or platelet, alpha granules contain proteins captured from the plasma via either fluid phase endocytosis (i.e., IgG and albumin) or receptor-mediated endocytosis (Harrison, P. and Cramer, E. M. (1993) Blood Rev. 7:52-62; Bouchard, B. A. and Tracy, P. B. (2001) Hematol. 8:263-269). Examples of the latter include fibrinogen, endocytosed by platelet glycoprotein IlbIla, and clotting factor V (fV) which is endocytosed via an as yet unknown receptor (Handagama, P. et al. (1993) Blood 82:135-138; Camire, R. M. et al. (1998) Blood 92:3035-3041). Upon platelet activation a highly reactive platelet surface facilitates thrombin formation by providing lipid cofactors and facilitating high local concentrations clotting factors including fVIII, fVIIIa, fX, fXa, fV and fVa (Bouchard, B. A. and Tracy, P. B. (2001) Hematol. 8:263-269). These observations suggest the possibility that TLT-1 may serve to capture, package, and/or sequester intermediates of thrombin formation in anticipation of activation. Upon activation TLT-1 along with its cargo are rapidly made available at the cell surface.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule which encodes a TLT-1 polypeptide, or a complement thereof, wherein the TLT-1 polypeptide can modulate platelet function.

2. The nucleic acid molecule of claim 1, wherein the TLT-1 polypeptide is membrane-bound, or a complement thereof.

3. The nucleic acid molecule of claim 1, wherein the TLT-1 polypeptide is a soluble TLT-1 extracellular domain, or a complement thereof.

4. The nucleic acid molecule of claim 1, which encodes a murine TLT-1 polypeptide, or a complement thereof.

5. The nucleic acid molecule of claim 1, which encodes a human TLT-1 polypeptide, or a complement thereof.

6. An isolated nucleic acid molecule comprising the nucleotide sequence set forth in SEQ ID NO:1, 3, 4, 6, 18, 20, 21, 23, 24, or 26, or a complement thereof.

7. An isolated nucleic acid molecule which encodes a polypeptide comprising the amino acid sequence set forth in SEQ ID NO:2, 5, 19, 22, or 25, or a complement thereof.

8. An isolated nucleic acid molecule selected from the group consisting of:
(a) a nucleic acid molecule comprising a nucleotide sequence which is at least about 97% identical to the nucleotide sequence of SEQ ID NO:1 or 3, or a complement thereof;
(b) a nucleic acid molecule comprising a nucleotide sequence which is at least about 60% identical to the nucleotide sequence of SEQ ID NO:1, 3, 4, 6, 18, 20, 21, 23, 24, or 26, or a complement thereof, or a complement thereof;
(c) a nucleic acid molecule comprising at least 487 nucleotides of SEQ ID NO:1 or 3, or a complement thereof;
(d) a nucleic acid molecule comprising at least 336 nucleotides of SEQ ID NO:4 or 6, or a complement thereof;
(e) a nucleic acid molecule comprising at least 30 nucleotides of SEQ ID NO:1, 3, 4, 6, 18, 20, 21, 23, 24, and 26, or a complement thereof
(f) a nucleic acid molecule which encodes a polypeptide comprising an amino acid sequence which is at least about 99% identical to the amino acid sequence of SEQ ID NO:2, or a complement thereof;
(g) a nucleic acid molecule which encodes a polypeptide comprising an amino acid sequence which is at least about 81% identical to the amino acid sequence of SEQ ID NO:5, or a complement thereof;
(h) a nucleic acid molecule which encodes a polypeptide comprising an amino acid sequence which is at least about 60% identical to the amino acid sequence of SEQ ID NO:2, 5, 19, 22, or 25, or a complement thereof;
(i) a nucleic acid molecule which encodes at least 173 contiguous amino acid residues of SEQ ID NO:2, or a complement thereof;
(j) a nucleic acid molecule which encodes at least 111 contiguous amino acid residues of SEQ ID NO:5, or a complement thereof; and
(k) a nucleic acid molecule which encodes at least 10 contiguous amino acid residues of SEQ ID NO:2, 5, 19, 22, or 25, or a complement thereof.

9. An isolated nucleic acid molecule which hybridizes to a complement of the nucleic acid molecule of any one of claims 1-8 under stringent conditions.

10. An isolated nucleic acid molecule comprising the nucleic acid molecule of any one of claims 1-8 and a nucleotide sequence encoding a heterologous polypeptide.

11. A vector comprising the nucleic acid molecule of any one of claims 1-8.

12. The vector of claim 11, which is an expression vector.

13. A host cell transfected with the expression vector of claim 12.

14. A method of producing a polypeptide comprising culturing the host cell of claim 13 in an appropriate culture medium to, thereby, produce the polypeptide.

15. An isolated TLT-1 polypeptide, wherein the TLT-1 polypeptide can modulate platelet function.

16. The polypeptide of claim 15, wherein the TLT-1 polypeptide is membrane-bound.

17. The polypeptide of claim 15, wherein the TLT-1 polypeptide is a soluble TLT-1 extracellular domain.

18. The polypeptide of claim 15, which encodes a murine TLT-1 polypeptide.

19. The polypeptide of claim 15, which encodes a human TLT-1 polypeptide.

20. An isolated polypeptide comprising the amino acid sequence of SEQ ID NO:2, 5, 19, 22, or 25.

21. An isolated polypeptide selected from the group consisting of:
(a) a polypeptide comprising at least 173 contiguous amino acid residues of SEQ ID NO:2;
(b) a polypeptide comprising at least 111 contiguous amino acid residues of SEQ ID NO:5;
(c) a polypeptide comprising at least 10 contiguous amino acid residues of SEQ ID NO:2, 5, 19, 22, or 25.
(d) a polypeptide which is encoded by a nucleic acid molecule comprising a nucleotide sequence which is at least about 97% identical to the nucleotide sequence of SEQ ID NO:1 or 3;
(e) a polypeptide which is encoded by a nucleic acid molecule comprising a nucleotide sequence which is at least about 60% identical to the nucleotide sequence of SEQ ID NO:1, 3, 4, 6, 18, 20, 21, 23, 24, and 26;
(f) a polypeptide comprising an amino acid sequence which is at least about 99% identical to the amino acid sequence of SEQ ID NO:2;
(g) a polypeptide comprising an amino acid sequence which is at least about 81% identical to the amino acid sequence of SEQ ID NO:5; and
(h) a polypeptide comprising an amino acid sequence which is at least about 60% identical to the amino acid sequence of SEQ ID NO:2, 5, 19, 22, or 25.

22. The polypeptide of any one of claims 15-21, further comprising heterologous amino acid sequences.

23. An antibody which selectively binds to a polypeptide of any one of claims 15-21.

24. A method for detecting the presence of a polypeptide of any one of claims 15-21 in a sample comprising:
a) contacting the sample with a compound which selectively binds to the polypeptide; and
b) determining whether the compound binds to the polypeptide in the sample to thereby detect the presence of a polypeptide of any one of claims 15-21 in the sample.

25. The method of claim 24, wherein the compound which binds to the polypeptide is an antibody.

26. A kit comprising a compound which selectively binds to a polypeptide of any one of claims 15-21 and instructions for use.

27. A method for detecting the presence of a nucleic acid molecule of any one of claims 1-8 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of any one of claims 1-8 in the sample.

28. The method of claim 27, wherein the sample comprises mRNA molecules and is contacted with a nucleic acid probe.

29. A kit comprising a compound which selectively hybridizes to a nucleic acid molecule of any one of claims 1-8 and instructions for use.

30. A method for identifying a compound which binds to a polypeptide of any one of claims 15-21 comprising:
a) contacting the polypeptide, or a cell expressing the polypeptide with a test compound; and
b) determining whether the polypeptide binds to the test compound.

31. The method of claim 30, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for TLT-1 activity.

32. A method for modulating the activity of a polypeptide of any one of claims 15-21 comprising contacting the polypeptide, or a cell expressing the polypeptide, with a compound which binds to the polypeptide in a sufficient concentration to modulate the activity of the polypeptide.

33. A method for identifying a compound which modulates the activity of a polypeptide of any one of claims 15-21 comprising:
a) contacting a polypeptide of any one of claims 15-21 with a test compound; and
b) determining the effect of the test compound on the activity of the polypeptide to thereby identify a compound which modulates the activity of the polypeptide.

34. A method for identifying a compound capable of treating a disorder selected from the group consisting of septic shock, cancer, infectious disease, stroke, heart disease, myocardial infarction, arteriosclerosis, clotting disorders, bleeding disorders, platelet insufficiency, and a TLT-1 associated disorder, comprising assaying the ability of the compound to modulate TLT-1 nucleic acid expression or TLT-1 polypeptide activity, thereby identifying a compound capable of treating a disorder selected from the group consisting of septic shock, cancer, infectious disease, stroke, heart disease, myocardial infarction, arteriosclerosis, clotting disorders, bleeding disorders, platelet insufficiency, and a TLT-1 associated disorder.

35. A method for treating a subject having a disorder selected from the group consisting of septic shock, cancer, infectious disease, stroke, heart disease, myocardial infarction, arteriosclerosis, clotting disorders, bleeding disorders, platelet insufficiency, and a TLT-1 associated disorder, comprising administering to the subject a therapeutically effective amount of a TLT-1 modulator, thereby treating said subject having a disorder selected from the group consisting of septic shock, cancer, infectious disease, stroke, heart disease, myocardial infarction, arteriosclerosis, clotting disorders, bleeding disorders, platelet insufficiency, and a TLT-1 associated disorder.

36. The method of claim 35, wherein the TLT-1 modulator is a TLT-1 polypeptide.

37. The method of claim 36, wherein the TLT-1 polypeptide is a soluble TLT-1 extracellular domain.

38. The method of claim 35, wherein the TLT-1 modulator is an antibody that selectively binds to TLT-1.


SEQUENCE LISTING (26 sequences)

SEQ ID NO: 1 - DNA, 1220 bp, Mus musculus
agaacctactactgcccagccatggactgctacctgctgctgctgctgctgctcctgggactagcaggccaaggctcagctgacagtcatcccgaggtgctacaggcaccggtggggtcatccattctagtgcagtgccactaccggctccaggatgtgagggctctcaaggtgtggtgccagttcttgcaggaaggctgccacccactagtgacctcagcggtggaccgaagagctccgggaaacgggcgcatattcctcactgacctgggtggggggctcctgcaggtggaaatggtgaccctgcaggaggaggacacaggggagtatggttgtgtggtggagggagcggcaggaccccagaccctgcatagggtctccctgttggttcttccaccagtccctggcccaagagagggggaggaagcagaggacgagaaagaaacctatagaatcggaaccggaagtctgctcgaggacccctccttggacccttccgcgagtgctggtcctcacgagttcagacggcgtgagaacaggtgtcagaagcagtgtatccccctgatctggggtgctgtgctcctgttggccctggtggtggtggctgtggtgatatttgctgtgatggccagaaagaaagggaacaggcttgttgtctgtggcccgtcccagagcactggagttccaggaatggaccctccctcagcagcccaccgtagcagtgactcgggactaccctcggacattccacatgtgaggctcgactcaccgccttcctttgactctatctacacaggctcctctcttgatccaccatcaagcgaacccccagctccaccctcacagccccctctgcctcctaaggtcctgatgtcctccaagtctgtgacatatgccacagttgtcttcccaggaggggacaaaggtaaaatagcctcctgtgagccagttcaggacccaccaaacagtcaaactccacccagtaaataagagtacactttaatttattactcttgggatcacccctggggaattctctgcagcccggccaactagctctgccttttatgcccagagtatgtcagcgcctggtgactcagggctccaatctggtatcttggtttggtgcagtcagggagtggtgctccgatatgattggggtgtttggggaaagaattcttttctgctgcttcccggtgctgggtctaccgtcattacat

SEQ ID NO: 2 - protein, 322 aa, Mus musculus
MDCYLLLLLLLLGLAGQGSADSHPEVLQAPVGSSILVQCHYRLQDVRALKVWCQFLQEGCHPLVTSAVDRRAPGNGRIFLTDLGGGLLQVEMVTLQEEDTGEYGCVVEGAAGPQTLHRVSLLVLPPVPGPREGEEAEDEKETYRIGTGSLLEDPSLDPSASAGPHEFRRRENRCQKQCIPLIWGAVLLLALVVVAVVIFAVMARKKGNRLVVCGPSQSTGVPGMDPPSAAHRSSDSGLPSDIPHVRLDSPPSFDSIYTGSSLDPPSSEPPAPPSQPPLPPKVLMSSKSVTYATVVFPGGDKGKIASCEPVQDPPNSQTPPSK

SEQ ID NO: 3 - DNA, 966 bp, Mus musculus
atggactgctacctgctgctgctgctgctgctcctgggactagcaggccaaggctcagctgacagtcatcccgaggtgctacaggcaccggtggggtcatccattctagtgcagtgccactaccggctccaggatgtgagggctctcaaggtgtggtgccagttcttgcaggaaggctgccacccactagtgacctcagcggtggaccgaagagctccgggaaacgggcgcatattcctcactgacctgggtggggggctcctgcaggtggaaatggtgaccctgcaggaggaggacacaggggagtatggttgtgtggtggagggagcggcaggaccccagaccctgcatagggtctccctgttggttcttccaccagtccctggcccaagagagggggaggaagcagaggacgagaaagaaacctatagaatcggaaccggaagtctgctcgaggacccctccttggacccttccgcgagtgctggtcctcacgagttcagacggcgtgagaacaggtgtcagaagcagtgtatccccctgatctggggtgctgtgctcctgttggccctggtggtggtggctgtggtgatatttgctgtgatggccagaaagaaagggaacaggcttgttgtctgtggcccgtcccagagcactggagttccaggaatggaccctccctcagcagcccaccgtagcagtgactcgggactaccctcggacattccacatgtgaggctcgactcaccgccttcctttgactctatctacacaggctcctctcttgatccaccatcaagcgaacccccagctccaccctcacagccccctctgcctcctaaggtcctgatgtcctccaagtctgtgacatatgccacagttgtcttcccaggaggggacaaaggtaaaatagcctcctgtgagccagttcaggacccaccaaacagtcaaactccacccagtaaa

SEQ ID NO: 4 - DNA, 936 bp, Homo sapiens
atgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccagcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagggaacaggcttggtgtctgtggccgattcctgagcagcagagtttcaggcatgaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcctaaggtcctggtctgctccaagcctgtgacatatgccacagtaatcttcccgggagggaacaagggtggagggacctcgtgtgggccagcccagaatccacctaacaatcagactccatccagctaa

SEQ ID NO: 5 - protein, 311 aa, Homo sapiens
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 6 - DNA, 933 bp, Homo sapiens
atgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccagcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagggaacaggcttggtgtctgtggccgattcctgagcagcagagtttcaggcatgaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcctaaggtcctggtctgctccaagcctgtgacatatgccacagtaatcttcccgggagggaacaagggtggagggacctcgtgtgggccagcccagaatccacctaacaatcagactccatccagc

SEQ ID NO: 7 - DNA, 20 bp, Artificial
  Primer
gagcttgaaggatgaggaag

SEQ ID NO: 8 - DNA, 20 bp, Artificial
  Primer
gctcctcctgtgaaatagac

SEQ ID NO: 9 - DNA, 28 bp, Artificial
  Primer
cccaagcttaacaccacggtgctgcagg

SEQ ID NO: 10 - DNA, 28 bp, Artificial
  Primer
cgcggatcctgactggacttaagctgta

SEQ ID NO: 11 - DNA, 20 bp, Artificial
  Primer
gttagcacaccaggaaggag

SEQ ID NO: 12 - DNA, 21 bp, Artificial
  Primer
ctgtttctcagagactccctg

SEQ ID NO: 13 - DNA, 20 bp, Artificial
  Primer
atggatggatttgtcacgac

SEQ ID NO: 14 - DNA, 20 bp, Artificial
  Primer
aaatccagccatcatcacag

SEQ ID NO: 15 - DNA, 19 bp, Artificial
  Primer
agaacctactactgcccag

SEQ ID NO: 16 - DNA, 21 bp, Artificial
  Primer
gccaatatgtaatgacggtag

SEQ ID NO: 17 - protein, 16 aa, Homo sapiens
CDVPHIRLDSPPSFDN

SEQ ID NO: 18 - DNA, 599 bp, Mus musculus
gcccttagaacctactactgcccagccatggactgctacctgctgctgctgctgctgctcctgggactagcaggccaaggctcagctgacagtcatcccgaggtgctacaggcaccggtggggtcatccattctagtgcagtgccactaccggctccaggatgtgagggctctcaaggtgtggtgccagttcttgcaggaaggctgccacccactagtgacctcagcggtggaccgaagagctccgggaaacgggcgcatattcctcactgacctgggtggggggctcctgcaggtggaaatggtgaccctgcaggaggaggacacaggggagtatggttgtgtggtggagggagcggcaggaccccagaccctgcatagggtctccctgttggttcttccaccagtccctggcccaagagagggggaggaagcagaggacgagaaagaaacctatagaatcggaaccggaagtctgctcgaggacccctccttggacccttccgcgagtgctggtcctcacgagttcagacggcgtgagaacagtatccccctgatctggggtgctgtgctcctgttggctctggtggtggtggaaaa

SEQ ID NO: 19 - protein, 190 aa, Mus musculus
MDCYLLLLLLLLGLAGQGSADSHPEVLQAPVGSSILVQCHYRLQDVRALKVWCQFLQEGCHPLVTSAVDRRAPGNGRIFLTDLGGGLLQVEMVTLQEEDTGEYGCVVEGAAGPQTLHRVSLLVLPPVPGPREGEEAEDEKETYRIGTGSLLEDPSLDPSASAGPHEFRRRENSIPLIWGAVLLLALVVVE

SEQ ID NO: 20 - DNA, 572 bp, Mus musculus
atggactgctacctgctgctgctgctgctgctcctgggactagcaggccaaggctcagctgacagtcatcccgaggtgctacaggcaccggtggggtcatccattctagtgcagtgccactaccggctccaggatgtgagggctctcaaggtgtggtgccagttcttgcaggaaggctgccacccactagtgacctcagcggtggaccgaagagctccgggaaacgggcgcatattcctcactgacctgggtggggggctcctgcaggtggaaatggtgaccctgcaggaggaggacacaggggagtatggttgtgtggtggagggagcggcaggaccccagaccctgcatagggtctccctgttggttcttccaccagtccctggcccaagagagggggaggaagcagaggacgagaaagaaacctatagaatcggaaccggaagtctgctcgaggacccctccttggacccttccgcgagtgctggtcctcacgagttcagacggcgtgagaacagtatccccctgatctggggtgctgtgctcctgttggctctggtggtggtggaaaa

SEQ ID NO: 21 - DNA, 1205 bp, Mus musculus
agaacctactactgcccagccatggactgctacctgctgctgctgctgctgctcctgggactagcaggccaaggctcagctgacagtcatcccgaggtgctacaggcaccggtggggtcatccattctagtgcagtgccactaccggctccaggatgtgagggctctcaaggtgtggtgccagttcttgcaggaaggctgccacccactagtgacctcagcggtggaccgaagagctccgggaaacgggcgcatattcctcactgacctgggtggggggctcctgcaggtggaaatggtgaccctgcaggaggaggacacaggggagtatggttgtgtggtggagggagcggcaggaccccagaccctgcatagggtctccctgttggttcttccaccagtccctggcccaagagagggggaggaagcagaggacgagaaagaaacctatagaatcggaaccggaagtctgctcgaggacccctccttggacccttccgcgagtgctggtcctcacgagttcagacggcgtgagaacagtatccccctgatctggggtgctgtgctcctgttggccctggtggtggtggctgtggtgatatttgctgtgatggccagaaagaaagggaacaggcttgttgtctgtggcccgtcccagagcactggagttccaggaatggaccctccctcagcagcccaccgtagcagtgactcgggactaccctcggacattccacatgtgaggctcgactcaccgccttcctttgactctatctacacaggctcctctcttgatccaccatcaagcgaacccccagctccaccctcacagccccctctgcctcctaaggtcctgatgtcctccaagtctgtgacatatgccacagttgtcttcccaggaggggacaaaggtaaaatagcctcctgtgagccagttcaggacccaccaaacagtcaaactccacccagtaaataagagtacactttaatttattactcttgggatcacccctggggaattctctgcagcccggccaactagctctgccttttatgcccagagtatgtcagcgcctggtgactcagggctccaatctggtatcttggtttggtgcagtcagggagtggtgctccgatatgattggggtgtttggggaaagaattcttttctgctgcttcccggtgctgggtctaccgtcattacat

SEQ ID NO: 22 - protein, 317 aa, Mus musculus
MDCYLLLLLLLLGLAGQGSADSHPEVLQAPVGSSILVQCHYRLQDVRALKVWCQFLQEGCHPLVTSAVDRRAPGNGRIFLTDLGGGLLQVEMVTLQEEDTGEYGCVVEGAAGPQTLHRVSLLVLPPVPGPREGEEAEDEKETYRIGTGSLLEDPSLDPSASAGPHEFRRRENSIPLIWGAVLLLALVVVAVVIFAVMARKKGNRLVVCGPSQSTGVPGMDPPSAAHRSSDSGLPSDIPHVRLDSPPSFDSIYTGSSLDPPSSEPPAPPSQPPLPPKVLMSSKSVTYATVVFPGGDKGKIASCEPVQDPPNSQTPPSK

SEQ ID NO: 23 - DNA, 951 bp, Mus musculus
atggactgctacctgctgctgctgctgctgctcctgggactagcaggccaaggctcagctgacagtcatcccgaggtgctacaggcaccggtggggtcatccattctagtgcagtgccactaccggctccaggatgtgagggctctcaaggtgtggtgccagttcttgcaggaaggctgccacccactagtgacctcagcggtggaccgaagagctccgggaaacgggcgcatattcctcactgacctgggtggggggctcctgcaggtggaaatggtgaccctgcaggaggaggacacaggggagtatggttgtgtggtggagggagcggcaggaccccagaccctgcatagggtctccctgttggttcttccaccagtccctggcccaagagagggggaggaagcagaggacgagaaagaaacctatagaatcggaaccggaagtctgctcgaggacccctccttggacccttccgcgagtgctggtcctcacgagttcagacggcgtgagaacagtatccccctgatctggggtgctgtgctcctgttggccctggtggtggtggctgtggtgatatttgctgtgatggccagaaagaaagggaacaggcttgttgtctgtggcccgtcccagagcactggagttccaggaatggaccctccctcagcagcccaccgtagcagtgactcgggactaccctcggacattccacatgtgaggctcgactcaccgccttcctttgactctatctacacaggctcctctcttgatccaccatcaagcgaacccccagctccaccctcacagccccctctgcctcctaaggtcctgatgtcctccaagtctgtgacatatgccacagttgtcttcccaggaggggacaaaggtaaaatagcctcctgtgagccagttcaggacccaccaaacagtcaaactccacccagtaaa

SEQ ID NO: 24 - DNA, 907 bp, Homo sapiens
cagtgtggtggaattgcccttatgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccggcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcataaggtcctggtctgctccaagcctgtgacatatgccacagtaatcttcccgggagggaacaagggtggagggacctcgtgtgggccagcccagaatccacctaacaatcagactccatccagcaagggc

SEQ ID NO: 25 - protein, 199 aa, Homo sapiens
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQESLLSGPPRQ

SEQ ID NO: 26 - DNA, 597 bp, Homo sapiens
atgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccggcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagaatccctcctcagtggtccaccacgtcag